(19) United States
(12) Patent Application Publication
(10) Pub. No.: US 2026/0206779 A1
(43) Pub. Date: Jul. 23, 2026

(54) ALTERNATIVE DAIRY FOOD PRODUCTS COMPRISING RECOMBINANT DAIRY INGREDIENT(S)

(71) Applicant: Re-Milk Ltd., Ness Ziona (IL)

(72) Inventors: Noa BEN YISHAI, Ness Ziona (IL); Liam SHARON, Ness Ziona (IL)

(21) Appl. No.: 19/139,712

(22) PCT Filed: Dec. 14, 2023
(86) PCT No.: PCT/IL2023/051268

§ 371 Date: Jun. 16, 2025

Related U.S. Application Data
(60) Provisional application No. 63/433,457, filed on Dec. 18, 2022.
(60) Provisional application No. 63/500,624, filed on May 8, 2023.

(51) Int. Cl. A23C 9/123; A23C 9/127; A23C 9/13; A23C 11/06; A23C 20/00; C07K 14/47

(52) U.S. Cl. CPC A23C 9/1236; A23C 9/127; A23C 9/1322; A23C 11/065; A23C 20/00; C07K 14/4717

(57) ABSTRACT

The present invention provides alternative food products with an improved flavour comprising at least one recombinant dairy ingredient, a yeast derived ingredient and a lactic acid derived ingredient as well as methods of preparation thereof. Specifically, methods for preparing such alternative food products comprising addition of addition of yeasts compositions, threonine, or a combination thereof, to lactic acid bacteria, are provided. Specific yoghurt or cream cheese compositions comprising recombinant beta lactoglobulin and yeast extract or yeast cells, fermented with lactic acid bacteria are disclosed.

### FIELD OF THE INVENTION

The present invention relates to alternative dairy food products comprising at least one recombinant dairy ingredient and at least one yeast-derived composition or ingredient, and to methods of preparation thereof.

### BACKGROUND OF THE INVENTION

Lactobacillus are bacteria that produce lactic acid as the major metabolic end product of carbohydrate fermentation, giving them the common name lactic acid bacteria (LAB). Production of lactic acid has linked LAB with food fermentations, as acidification inhibits the growth of spoilage agents. Lactic acid and other metabolic products contribute to the organoleptic and textural profile of food items such as yogurts and cheeses. The industrial importance of the LAB is further evidenced by their “generally recognized as safe” (GRAS) status, due to their ubiquitous appearance in dairy foods.

Although commercial LAB mixtures are composed of different LAB strains, all of the LAB strains have been selected due to their capability to flourish and use mammalian milk, especially of cows, sheep, and goats. Milk is the primary source of nutrition for young mammals before they are able to digest solid food, and thus includes many nutrients, including lipids, proteins, salts, minerals, vitamins, and carbohydrates. LAB use (ferment) these milk nutrients to give each dairy food its characteristic taste and smell, while acidifying the product.

Although LAB are somewhat capable of fermenting alternative dairy products which include recombinant dairy ingredients, the process is not as effective as in milk-derived dairy products. Therefore, there is a need in the field of alternative (non-animal) food products, specifically in the field of LAB-fermented alternative dairy foods, for methods and compositions to allow LAB to grow, contribute to flavor development, and acidify the alternative dairy food product.

### SUMMARY OF THE INVENTION

The present invention is based on the unexpected observation that the addition of yeast-derived extract during the preparation of alternative dairy products allowed obtaining fermented dairy products with enhanced flavor and acidity.

According to one aspect, the present invention provides a method comprising the steps of: (a) producing a composition comprising a recombinant dairy ingredient, a yeast composition and lactic-acid-bacteria (LAB), thereby obtaining an alternative dairy product; and; (b) optionally, LAB-fermenting the alternative dairy product obtained in step (a), thereby obtaining a LAB-fermented alternative dairy product.

In some examples, step (a) comprises the steps of: (i) providing a composition comprising a recombinant dairy ingredient; (ii) optionally, homogenizing the composition of step (i); (iii) optionally, pasteurizing the composition of step (i) or step (ii); (iv) adding a yeast composition to the composition obtained in any one of the preceding steps; and (v) adding lactic-acid-bacteria (LAB) to the composition obtained in step (iv), thereby obtaining an alternative dairy product. The terms “producing a composition” and “preparing a composition” may be used interchangeably and may encompass mixing the components forming the composition in any order, unless stated otherwise.

In some examples, the yeast composition comprises at least one of: (i) a yeast-derived amino-acid, peptide, or protein; (ii) a yeast-derived carbohydrate; (iii) a yeast-derived lipid; (iv) a yeast-derived vitamin; (v) a yeast-derived mineral; and (vi) yeast cells.

In other examples, the yeast composition comprises a mixture of yeast-derived amino-acids, peptides, proteins, carbohydrates, vitamins, and minerals.

In some examples, the yeast composition is selected from the group consisting of a yeast extract, soluble fraction of yeast cells and yeast cells, and any combination thereof.

In some of the above examples, the yeast composition constitutes from about 0.001 to about 1 wt % of the alternative dairy product or the LAB-fermented alternative dairy product. In any one of the above examples, the method comprises adding from about 0.001 to about 1 wt % of in step (iv).

According to some examples, the method further comprises adding L-threonine at step (a). According to some examples, the method further comprises adding L-threonine at any one of the steps (i)-(v). According to some examples, the method further comprises adding L-threonine at any one of the steps (i), (iv) and (v). In one example, the method comprises adding from about 0.01 to about 0.22 wt % of L-threonine.

According to some examples, the recombinant dairy ingredient is selected from the group consisting of: a recombinant dairy protein; a recombinant dairy fat; and a recombinant dairy carbohydrate. According to some examples, the recombinant dairy ingredient is a recombinant dairy protein. In one example, the recombinant dairy protein constitutes at least 10% of all proteins in the resulting alternative dairy product. In some examples, the recombinant dairy protein is a recombinant whey protein. In some examples, the recombinant dairy protein is β-Lactoglobulin (BLG). In some examples, the recombinant dairy protein is the only dairy protein in the alternative dairy product. In some examples, the recombinant dairy protein is the only whey protein in the alternative dairy product. In some examples, the recombinant dairy protein is the only protein in the alternative dairy product.

In some examples, the method comprises homogenization in step (ii), a pasteurization in step (iii), or both.

In some examples, the LAB is selected from the group consisting of: Lactobacillus Bulgaricus, Streptococcus Thermophilus, Streptococcus Group N1, Leuconostoc, Lactobacillus Acidophilus, Lactobacillus Casei, Lactobacillus Paracasei, Bifidobacterium Lactis, and any combination thereof.

According to any one of the above examples, the method comprises LAB fermentation in step (b), thereby obtaining a LAB-fermented alternative dairy product.

Thus, in some examples, the present invention provides a method comprising the steps of: (a) producing a composition comprising a recombinant dairy ingredient, a yeast composition and lactic-acid-bacteria (LAB), thereby obtaining an alternative dairy product; and; (b) LAB fermenting the alternative dairy product obtained in step (a), thereby obtaining a LAB-fermented alternative dairy product.

According to any one of the above examples, the method is for at least one of: (i) enabling or improving LAB fermentation of an alternative dairy product; (ii) improving the flavor of a fermented alternative dairy product; (iii) increasing the sourness of a fermented alternative dairy product; (iv) decreasing the pH of a fermented alternative dairy product; (v) reducing the off flavor of a fermented alternative dairy product; (vi) shortening the time of acidification of an alternative dairy product; (vii) increasing the acetaldehyde level of a fermented alternative dairy product; and/or (viii) decreasing the acetic acid level of a fermented alternative dairy product.

According to any one of the above examples the method is for any combination of the following: (i) improving the flavor of a fermented alternative dairy product; (ii) increasing the sourness of a fermented alternative dairy product; (iii) decreasing the pH of a fermented alternative dairy product; (iv) reducing the off flavor of a fermented alternative dairy product; (v) shortening the time of acidification of an alternative dairy product; (vi) increasing the acetaldehyde level of a fermented alternative dairy product; and/or (vii) decreasing the acetic acid level of a fermented alternative dairy product.

According to any one of the above examples, the method further comprising adding (i) from 1% to 10% of a dairy protein; (ii) from 1% to 15% of a sugar; (iii) from 0% to 30% of an oil; (iv) from 0.1% to 3% of a flavoring salt; (v) from 0.01% to 0.5% of a calcium source; (vi) from 0.001% to 1% of LAB; and (vii) from 0.001% to 1% of a yeast composition.

According to some examples, the method comprises adding from 0.01% to 1% L-threonine.

According to some examples, the dairy protein is a recombinant dairy protein. According to some examples, the dairy protein is BLG, e.g., rBLG. According to some examples, the method comprises adding from 1 to 10% BLG. According to some examples, the BLG is a sole milk/dairy protein or a sole whey protein or a sole protein added.

According to some examples, the method comprises adding (i) from 2% to 8% of a dairy protein; (ii) from 1% to 5% of a sugar; (iii) from 2% to 25% of an oil; (iv) from 0.5% to 1.5% of a flavoring salt; (v) from 0.05% to 0.25% of a calcium source; (vi) from 0.01% to 0.1% of LAB; and (vii) from 0.01% to 0.1% of a yeast composition. According to some examples, the method comprises adding from 0.02% to 0.25% L-threonine.

According to some examples, the obtained alternative dairy product or the LAB-fermented alternative dairy product comprises (i) from 1% to 10% of a dairy protein; (ii) from 1% to 15% of a sugar; (iii) from 0% to 30% of an oil; (iv) from 0.1% to 3% of a flavoring salt; (v) from 0.01% to 0.5% of a calcium source; (vi) from 0.001% to 1% of LAB; and (vii) from 0.001% to 1% of a yeast composition or yeast-derived ingredient(s). According to some examples, the obtained alternative dairy product or the LAB-fermented alternative dairy product comprises (i) from 2% to 8% of a dairy protein; (ii) from 1% to 5% of a sugar; (iii) from 2% to 25% of an oil; (iv) from 0.5% to 1.5% of a flavoring salt; (v) from 0.05% to 0.25% of a calcium source; (vi) from 0.01% to 0.1% of LAB; and (vii) from 0.01% to 0.1% of a yeast composition or yeast-derived ingredient(s).

According to some examples, the alternative dairy product is selected from the group consisting of yogurt, cheese, cream cheese, buttermilk, sour cream, kefir, soured milk, Crème fraiche, and ryazhenka. According to some examples, the alternative dairy product is yogurt. According to some examples, the alternative dairy product is cream cheese.

According to some examples, the present invention provides an alternative dairy product obtainable or obtained by the method according to any one of the above examples. According to one example, the alternative dairy product comprises: (i) a recombinant dairy ingredient; (ii) a yeast-derived ingredient; (iii) a lactic-acid-bacteria (LAB)-derived ingredient; and (iv) optionally, at least one of a LAB-fermentation-related ingredient, L-threonine and acetaldehyde.

According to another aspect, the present invention provides an alternative dairy product, comprising: (i) a recombinant dairy ingredient; (ii) a yeast-derived ingredient; (iii) a lactic-acid-bacteria (LAB)-derived ingredient; and (iv) optionally, at least one of a LAB-fermentation-related ingredient, L-threonine, and acetaldehyde.

According to some examples, the yeast-derived ingredient is selected from the group consisting of: (i) a yeast cell; (ii) yeast DNA; (iii) yeast RNA; (iv) a yeast-derived amino-acid, peptide or protein; (v) a yeast metabolite; and (vi) any combination of (i) to (v).

In other examples, the LAB-derived ingredient is selected from the group consisting of: (i) a LAB cell; (ii) LAB DNA; (iii) LAB RNA; (iv) a LAB-derived amino-acid, peptide or protein; (v) a LAB-derived metabolite; and (vi) any combination of (i) to (v).

In some examples, the alternative dairy product comprises at least one LAB-fermentation-related ingredient. According to some examples, the LAB-fermentation-related ingredient is selected from the group consisting of: lactic acid; a volatile compound selected from the group consisting of acetaldehyde, diacetyl, and acetoin; and any combination thereof. According to one example, the alternative dairy product comprises acetaldehyde. According to some examples, the alternative dairy product comprises from 10 to 40 ppm of acetaldehyde. According to one example, the alternative dairy product comprises from 15 to 30 ppm of acetaldehyde.

According to some of the above examples, the recombinant dairy ingredient is selected from the group consisting of: (i) a recombinant dairy protein; (ii) a recombinant dairy fat; and (iii) a recombinant dairy carbohydrate.

According to some examples, the recombinant dairy ingredient is selected from the group consisting of: a recombinant dairy protein; a recombinant dairy fat; and a recombinant dairy carbohydrate. According to some examples, the recombinant dairy ingredient is a recombinant dairy protein. In one example, the recombinant dairy protein constitutes at least 10% of all proteins in the resulting alternative dairy product. In some examples, the recombinant dairy protein is a recombinant whey protein. In some examples, the recombinant dairy protein is β-Lactoglobulin (BLG). In some examples, the recombinant dairy protein is the only dairy protein in the alternative dairy product. In some examples, the recombinant dairy protein is the only whey protein in the alternative dairy product. In some examples, the recombinant dairy protein is the only dairy protein in the alternative dairy product.

In some examples, the alternative dairy product comprises or the LAB-fermented alternative dairy product comprises (i) from 1% to 10% of a dairy protein; (ii) from 1% to 15% of a sugar; (iii) from 0% to 30% of an oil; (iv) from 0.1% to 3% of a flavoring salt; (v) from 0.01% to 0.5% of a calcium source; (vi) from 0.001% to 1% of LAB; and (vii) from 0.001% to 1% of a yeast composition or yeast derived ingredients. According to some examples, the alternative dairy product or the LAB-fermented alternative dairy product comprises (i) from 2% to 8% of a dairy protein; (ii) from 1% to 5% of a sugar; (iii) from 2% to 25% of an oil; (iv) from 0.5% to 1.5% of a flavoring salt; (v) from 0.05% to 0.25% of a calcium source; (vi) from 0.01% to 0.1% of LAB; and (vii) from 0.01% to 0.1% of a yeast composition or yeast derived ingredients.

In some examples, the alternative dairy product is selected from the group consisting of yogurt, cheese, cream cheese, buttermilk, sour cream, kefir, soured milk, crème fraiche, and ryazhenka. In some examples, the alternative dairy product is an alternative cream cheese. In some examples, the alternative dairy product is an alternative yogurt.

According to some examples, the present invention provides an alternative dairy product of the present invention obtainable or obtained by the methods of the present invention.

### BRIEF DESCRIPTION OF DRAWINGS

FIG. 1 shows flavor ranking of the alternative yogurt prepared with the addition of yeast extract (YE) NuCel® 545 MG at different concentrations in comparison to the reference (yogurt prepared from milk and shea fat).

FIG. 2 shows flavor ranking of the alternative yogurt prepared with the addition of yeast extract NuCel® 581 PW at different concentrations in comparison to the reference (yogurt prepared from milk and shea fat).

FIG. 3 shows flavor ranking of the alternative yogurt prepared with the addition of yeast extract NuCel® 582 MG at different concentrations in comparison to the reference (yogurt prepared from milk and shea fat).

FIG. 4 shows flavor ranking of the alternative yogurt prepared with the addition of yeast extract NuCel® 751 MG at different concentrations in comparison to the reference (yogurt prepared from milk and shea fat).

FIG. 5 shows flavor ranking of the alternative yogurt prepared with the addition of 0.05 wt % of different yeast extracts in comparison to the reference (yogurt prepared from milk and shea fat).

FIG. 6 shows flavor ranking of the alternative yogurt prepared with the addition of 0.07 wt % of different yeast extracts in comparison to the reference (yogurt prepared from milk and shea fat).

FIG. 7 shows flavor ranking of the alternative cream cheese prepared with the addition of yeast extract 581 at different concentrations in comparison to the reference (cream cheese prepared from whey protein isolate and shea fat).

FIG. 8 shows acidification curves of alternative cream cheese formulations.

FIG. 9 shows the effect of the addition of L-threonine in various concentrations during the preparation of yogurt on the yogurt flavor of the final product.

FIG. 10 shows the effect of the addition of L-threonine during the preparation of yogurt on the amount of different volatile compounds in the final product.

FIG. 11 shows the effect of the addition of L-threonine during the preparation of yogurt on the amount of acetic acid in the final product.

FIG. 12 shows an acidification curve of alternative yogurt with or without 0.02% yeast extract.

### DETAILED DESCRIPTION OF THE INVENTION

Unless otherwise defined, all technical and/or scientific terms used herein have the same meaning as commonly understood by one of ordinary skill in the art to which the invention pertains. In case of conflict, the patent specification, including definitions, will control.

The present invention provides methods for preparing alternative (non-animal) dairy food products and fermented alternative dairy food products comprising a recombinant dairy ingredient, which have rheological and organoleptic characteristics similar to their corresponding animal-based dairy products. As proper fermentation of lactic acid bacteria (LAB) is at least partly responsible for some of these characteristics, it is essential to craft alternative dairy food products such that they allow LAB to flourish.

It was found that LAB do not sufficiently ferment recombinant dairy ingredients and thus do not fully contribute to flavor development and acidification in alternative dairy food products. The Inventors have further found that the addition of different combinations of amino acids, minerals, vitamins and metabolites to alternative dairy food products also failed to allow proper LAB fermentation.

It has been found that the addition of yeast cells or yeast extracts (the soluble cell contents of yeast without the cell walls) during the process of preparation of alternative dairy food products allows proper and efficient LAB fermentation, acidification, and characteristic flavor development of the alternative dairy products such as cream cheese and yogurt. In addition, the pH of the fermented alternative dairy products was close to traditional dairy products. Another advantage of adding yeast cells or extracts is that the time required for reaching the desired pH, as well as the time needed to develop characteristic tastes of fermented dairy products, is reduced. These effects consequently reduce the cost of production of alternative dairy products. It is hereby demonstrated that the addition of whole yeast cells (such as Saccharomyces cerevisiae) or yeast cell extracts provides significant impacts on pH reduction and flavor development of alternative dairy products. It was further surprisingly found that the addition of small amounts of amino acid L-threonine further improves the taste of the fermented product. Interestingly, increasing the concentration of L-threonine above 0.25 wt % negatively affected this property.

According to one aspect, the present invention provides a method comprising the steps of: (a) producing a composition comprising a recombinant dairy ingredient, a yeast composition and lactic-acid-bacteria (LAB), thereby obtaining an alternative dairy product; and (b) optionally, LAB fermenting the alternative dairy product obtained in step (a), thereby obtaining a LAB-fermented alternative dairy product.

According to some embodiment, the present invention provides a method comprising the steps of: (a) producing a composition comprising a recombinant dairy ingredient, a yeast composition and lactic-acid-bacteria (LAB); and (b) LAB fermenting the alternative dairy product obtained in step (a), thereby obtaining a LAB-fermented alternative dairy product.

According to some embodiments, the method of the present invention is for at least one of: (i) enabling or improving LAB fermentation of an alternative dairy product; (ii) improving the flavor of a fermented alternative dairy product; (iii) increasing the sourness of a fermented alternative dairy product; (iv) decreasing the pH of a fermented alternative dairy product; (v) reducing the off flavor of a fermented alternative dairy product; (vi) shortening the acidification time of a fermented alternative dairy product; (vii) increasing the acetaldehyde level of a fermented alternative dairy product; and (viii) decreasing the acetic acid level of a fermented alternative dairy product.

According to some embodiments, the method is for any combination of: (i) enabling or improving LAB fermentation of an alternative dairy product; (ii) improving the flavor of a fermented alternative dairy product; (iii) increasing the sourness of a fermented alternative dairy product; (iv) decreasing the pH of a fermented alternative dairy product; (v) reducing the off flavor of a fermented alternative dairy product; (vi) shortening the acidification time of a fermented alternative dairy product; (vii) increasing the acetaldehyde level of a fermented alternative dairy product; and (viii) decreasing the acetic acid level of a fermented alternative dairy product.

According to some embodiments, the method is for enabling or improving LAB fermentation of an alternative dairy product. According to some embodiments, the method is for improving the flavor of a fermented alternative dairy product. According to some embodiments, the method is for increasing the sourness of a fermented alternative dairy product. According to some embodiments, the method is for decreasing the pH of a fermented alternative dairy product. According to some embodiments, the method is for reducing the off flavor of a fermented alternative dairy product. According to some embodiments, the method is for shortening the acidification time of a fermented alternative dairy product. The terms “shortening the acidification time of a fermented alternative dairy product” and “shortening the time of acidification of an alternative dairy product” may be used interchangeably. According to some embodiments, the method is for increasing the acetaldehyde level of a fermented alternative dairy product. According to some embodiments, the method is for decreasing the acetic acid level of a fermented alternative dairy product. According to some embodiments, the method is for improving the flavor of a fermented alternative dairy product and for increasing the sourness of a fermented alternative dairy product. According to some embodiments, the method is for improving the flavor of a fermented alternative dairy product and for reducing the off flavor of a fermented alternative dairy product. According to some embodiments, the method is for improving the flavor of a fermented alternative dairy product, for increasing the sourness of a fermented alternative dairy product and for reducing the off flavor of a fermented alternative dairy product. According to some embodiments, the method is further for decreasing the pH of a fermented alternative dairy product According to some embodiments, the method is for increasing the acetaldehyde level of a fermented alternative dairy product and for improving the flavor of a fermented alternative dairy product. According to some embodiments, the method is for increasing the acetaldehyde level of a fermented alternative dairy product and for decreasing the acetic acid level of a fermented alternative dairy product.

In some embodiments, L-threonine may be added instead of or in addition to the yeast composition. Thus, according to some embodiments, the present invention provides a method comprising the steps of: (a) producing a composition comprising a recombinant dairy ingredient, lactic-acid-bacteria (LAB) and (i) a yeast composition, (ii) L-threonine, or (iii) both a yeast composition and L-threonine, thereby obtaining an alternative dairy product; and (b) optionally, LAB-fermenting the alternative dairy product obtained in step (a), thereby obtaining a LAB-fermented alternative dairy product.

The method of the present invention provides a method for preparing an alternative dairy product. In addition, when fermentation takes place, the present invention provides a method for preparing a fermented alternative dairy product.

According to some embodiment, step (a) may be further divided into several steps. Therefore, according to some embodiments, step (a) comprises the steps of: (i) providing a composition comprising a recombinant dairy ingredient; (ii) optionally, homogenizing the composition of step (i); (iii) optionally, pasteurizing the composition of step (i) or step (ii), if present; (iv) adding a yeast composition to the composition obtained in any one of the preceding steps; and (v) adding lactic-acid-bacteria (LAB) to the composition obtained in step (iv), thereby obtaining an alternative dairy product.

The order of the steps may vary. For example, in some embodiments, step (iv) may be performed before step (iii). In other embodiments, step (iv) and (v) may be performed in a single step.

According to some embodiments, the present invention provides a method for preparing an alternative dairy product, the method comprising the steps of: (I) providing a composition comprising a recombinant dairy ingredient; (II) adding a yeast composition and/or L-threonine to the composition obtained in step (I); (III) adding lactic-acid-bacteria (LAB) to the composition obtained in step (I) and/or step (II), thereby obtaining an alternative dairy product; and (IV) optionally, allowing the LAB to ferment the alternative dairy product obtained in step (III), thereby obtaining a LAB-fermented alternative dairy product.

According to some embodiments, the method comprises adding L-threonine in step (II). According to another embodiment, the method comprises adding a yeast composition in step (II). According to some embodiments, the method comprises adding L-threonine and a yeast composition in step (II).

According to some embodiments, the present invention provides a method for preparing an alternative dairy product, the method comprising the steps of: (i) providing a composition comprising a recombinant dairy ingredient; (ii) optionally, homogenizing the composition of step (i); (iii) optionally, pasteurizing the composition of step (i) or step (ii), if present; (iv) adding a yeast composition to the composition obtained in any one of the preceding steps; (v) adding lactic-acid-bacteria (LAB) to the composition obtained in step (iv), thereby obtaining an alternative dairy product; and (vi) optionally, allowing the LAB to ferment the alternative dairy product of step (v); thereby obtaining a LAB-fermented alternative dairy product. Step (b) of the aspect may also be referred to step (vi) in some detailed embodiments.

The term “yeast composition” refers to any composition comprising a yeast-derived component. The term “yeast composition” further refers to the well-known category of commercial products known as “yeast extract”, as well as to yeast cells, either in liquid or dried form, either viable or not. According to some embodiments, the yeast composition comprises a yeast extract. According to some embodiments, the yeast composition comprises yeast cells, e.g., whole yeast cells. According to some embodiments, the yeast composition comprises the soluble fraction of yeast cells, e.g., yeast cell cytoplasm. According to some embodiments, the yeast composition comprises yeast cells and a yeast extract. According to some embodiments, the yeast composition comprises yeast cells the soluble fraction of yeast cells, and a yeast extract. In general, the term “yeast-derived component” refers to any ingredient or component that is derived from, originated from, extracted from or obtained from yeast and preferably may be identified as such.

Without being limited to any particular theory, it is assumed that the addition of yeast composition allows the lactic acid bacteria to prosper and subsequently to confer the alternative dairy product with the desired flavor and acidity. Therefore, the method of the present invention comprises adding yeast extract at any one of the steps.

According to some embodiment, the yeast composition comprises yeast extract. According to one embodiment, the method comprises adding yeast extract at step (iv). According to one embodiment, the method comprises adding yeast extract at step (i) instead of step (iv). According to one embodiment, the method comprises adding yeast extract before performing step (iii), instead of step (iv). According to one embodiment, the method comprises adding yeast extract at step (v), together with LAB. According to other embodiments, the yeast composition comprises live yeast cells. Therefore, the method of the present invention provides adding yeasts at any one of the steps preceding the adding LAB. According to one embodiment, the method comprises adding yeast cells in step (iv). According to one embodiment, the method comprises adding yeast cells after step (ii) and before step (iii), instead of step (iv). According to some embodiments, the method comprises adding yeast cells in step (v). According to some embodiments, the method of the present invention provides adding yeast extract and yeast cells in step (iv). According to one embodiment, the method comprises adding yeast cells in step (v), together with LAB. According to some embodiments, the yeast composition constitutes at least 0.001 wt %, at least 0.01 wt % or at least 0.1 wt % of the alternative dairy product. According to some embodiments, the yeast composition constitutes up to 5 wt %, up to 3 wt %, up to 2 wt % or up to 1 wt % of the alternative dairy product. According to some embodiments, the yeast composition constitutes from about 0.001 to about 1 wt % of the final alternative dairy product. According to some embodiments, the yeast composition constitutes from about 0.01 to about 0.3 wt % of the final alternative dairy product. According to some embodiments, the yeast composition constitutes from about 0.01 to about 0.3 wt % of the final alternative dairy product before fermentation. According to some embodiments, the yeast composition constitutes from about 0.01 to about 0.3 wt % of the final alternative dairy product after fermentation. According to some embodiments, the yeast composition constitutes from about 0.01 to about 0.15 wt % of the final alternative dairy product. According to some embodiments, the yeast composition constitutes from about 0.01 to about 0.12 wt % of the final alternative dairy product. According to some embodiments, the yeast composition constitutes from about 0.012 to about 0.12 wt % of the final alternative dairy product. According to some embodiments, the yeast composition constitutes from about 0.01 to about 0.1 wt %, from about 0.01 to about 0.09 wt %, from about 0.02 to about 0.08 wt % of the final alternative dairy product. According to some embodiments, the yeast composition constitutes about 0.03 wt %, about 0.04 wt %, about 0.05 wt %, about 0.06 wt %, about 0.07 wt % or about 0.08 wt % of the final alternative dairy product.

In the context of a method of preparation, specifying the concentration of a compound in the final product contemplates that the compound is added in the amount that provides the specified concentration in the final product. As such, the term “a component X constitutes Y %” and the term “the method comprises adding Y % of a component X” may be used interchangeably and have the meaning that a component X is added in such an amount that its concentration in the final product is Y. Any person skilled in the art can perform the required calculation to determine the amount of X that has to be added such that the final concentration would be Y. Therefore, according to some embodiments, the method comprises adding a yeast composition in the concentration to result in the abovementioned concentrations in the final product. According to some embodiments, the method comprises adding from 0.001 to 1 wt %, from about 0.01 to about 0.1 wt %, from about 0.01 to about 0.09 wt %, from about 0.02 to about 0.08 wt % of a yeast composition of the final alternative dairy product. According to some embodiments, the method comprises adding about 0.03 wt %, about 0.04 wt %, about 0.05 wt %, about 0.06 wt %, about 0.07 wt % or about 0.08 wt % of a yeast composition of the final alternative dairy product.

According to some embodiments, the yeast extract is a commercially available yeast extract. According to some embodiments, the yeast extract is a yeast peptone obtained by the autolysis of a selected strain of yeast, especially grown on a molasses-based media. According to some embodiments, the yeast extract is selected from NuCel® 581 PW, NuCel® 545 MG, NuCel® 582 MG and NuCel® 751 MG. According to some embodiments, the yeast extract has CAS number 84604-16-0. According to some embodiments, the yeast extract has EC number: 283-294-5.

According to some embodiments, the yeast composition comprises at least one of: yeast-derived amino acids, yeast-derived peptides, yeast-derived proteins, yeast-derived carbohydrates, yeast-derived vitamins, yeast-derived minerals, yeast-derived lipids, yeast-derived membranes or mixtures thereof. According to some embodiments, the yeast composition comprises whole yeast cells. According to some embodiments, the yeast composition comprises the soluble fraction of yeast cells. According to some embodiments, the yeast composition comprises two or more of the followings: yeast-derived peptides, yeast-derived proteins, yeast-derived carbohydrates, yeast-derived vitamins, yeast-derived minerals, yeast-derived lipids and yeast-derived membrane or fragments thereof. According to some embodiments, the yeast composition comprises a mixture of yeast-derived peptides, yeast-derived proteins, and yeast-derived carbohydrates. According to some embodiments, the yeast composition comprises a mixture of yeast-derived peptides, proteins, and carbohydrates, vitamins, and minerals. Yeast-derived compounds may comprise signatures that are unique to yeasts such as post-translational modification, the existence of unique lipids or ratios between different compounds that are characteristic of yeasts.

According to some embodiments, the method comprises adding L-threonine. According to some embodiments, the method comprises adding from about 0.01 to about 0.24 wt % L-threonine. According to some embodiments, the method comprises adding from about 0.02 to about 0.2 wt % L-threonine. According to some embodiments, the method comprises adding from about 0.03 to about 0.18 wt % L-threonine. According to some embodiments, the method comprises adding from about 0.03 to about 0.15 wt % L-threonine. According to some embodiments, the method comprises adding about 0.03, 0.06, 0.09, 0.1, 0.11, 0.12, 0.13, 0.14, 0.15, 0.16, 0.17, 0.18, 0.19 or 0.2 wt % L-threonine. According to some embodiments, the method comprises adding less than about 0.3 wt % L-threonine. According to some embodiments, the method comprises adding less than about 0.25 wt % L-threonine. According to some embodiments, the method comprises adding from about 0.01 to less than about 0.25 wt % L-threonine. According to some embodiments, the L-threonine is added before the initiation of fermentation, e.g., in steps (i), (iv) or (v).

According to some embodiments, the method comprises adding L-threonine and a yeast composition. According to some embodiments, the method comprises adding L-threonine at step (a). According to some embodiments, the method comprises adding L-threonine at step (i). According to some embodiments, the method comprises adding L-threonine at step (iv). According to some embodiments, the method comprises adding L-threonine at step (v). According to some embodiments, the L-threonine and the yeast composition are added substantially simultaneously. According to some embodiments, the L-threonine and the yeast composition are added separately.

According to some embodiments, the method comprises adding from 0.001 to 1 wt % of yeast extract and from 0.01 to up to 0.25 wt % L-threonine. According to some embodiments, the method comprises adding from 0.001 to 1 wt %, from about 0.01 to about 0.1 wt %, from about 0.01 to about 0.09 wt %, from about 0.02 to about 0.08 wt % of a yeast composition and from about 0.02 to about 0.2 wt %, from about 0.03 to about 0.18 wt % or from about 0.03 to about 0.15 wt % L-threonine. According to some embodiments, the method comprises adding about 0.03, 0.06, 0.09, 0.1, 0.11, 0.12, 0.13, 0.14, 0.15, 0.16, 0.17, 0.18, 0.19 or 0.2 wt % L-threonine. According to some embodiments, the method comprises adding from about 0.01 to about 0.1 wt %, from about 0.01 to about 0.09 wt % of a yeast composition and from about 0.03 to about 0.15 wt % L-threonine or about 0.03, 0.06, 0.09, 0.1, 0.11, 0.12, 0.13, 0.14, 0.15, 0.16, 0.17, 0.18, 0.19 or 0.2 wt % L-threonine. According to some embodiment, the yeast composition comprises yeast extract, yeast cells or both.

It would be understood to any person of average skills in the art that due to commercial and/or production-line considerations, it may be beneficial to homogenize the composition after adding and mixing some or all components of the composition. Therefore, it is clear that the method of the present invention may comprise a step of homogenization after performing steps (i) and/or (iv) or after performing steps (i), (iv) and (v), or after performing steps (i), (iv) and (vi) thus producing a homogenous composition comprising the recombinant dairy ingredient and the yeast composition or producing a homogenous composition comprising the recombinant dairy ingredient, the yeast composition and the LAB, respectively. Such homogenized composition(s) may be further pasteurized.

Therefore, in certain embodiments, the method further comprises homogenizing the composition obtained in step (iv). In some embodiments, the method further comprises homogenizing the composition obtained in step (v). In other embodiments, the method further comprises homogenizing the composition obtained in step (vi). According to some embodiments, the method further comprises pasteurizing the composition obtained in any of steps (iv), (v) or (iv). According to some embodiments, the method further comprises pasteurizing the homogenized composition obtained as described above.

According to any one of the above embodiments, the method comprises homogenization in step (ii). The terms “homogenized” and “homogenization” are known to any person in the art, and generally refer to the process or to the product that passed the process of homogenization. Homogenization may be performed by any known method and/or device. According to some embodiments, the homogenization is performed in 1, 2, 3 or 4 stages. According to some embodiments, the homogenization is performed at from about 50 to about 400 bars. According to some embodiments, homogenization is performed for from 2 to 120 minutes. According to the principles of the present invention, any homogenization stage and any homogenization pressure found to homogenize the compositions and products of the present invention are included. According to some embodiments, homogenization may be performed in two steps. Non-limiting examples are stage homogenizing at 50 or 60 bar and then at 200 bars. According to some embodiments, the composition is heated prior to homogenization, e.g., heated up to 50° C. or up to 60° C. o up to 70° C.

According to any one of the above embodiments, the method comprises pasteurization in step (iii). According to some embodiments, the pasteurization is performed at a temperature of less than 100° C., at times, less than 90° C.; at times, less than 80° C. In some examples, during pasteurization, the composition is mildly heated, typically at a temperature between 50° C. and 100° C.; at times at a temperature of between 50° C. and 90° C., or at a temperature of between 50° C. and 80° C. According to some embodiments, the composition is heated at a temperature of between 85° C. and 95° C. In some examples, pasteurization is carried out at a temperature of about 90° C. for several minutes. In some examples, pasteurization is carried out at a temperature range of 85° C. and 95° C., at times, at a temperature range of between about 87° C. and 93° C. According to some embodiments, the duration of pasteurization is typically between 1 and about 10 minutes, at times, between about 2 and 9 minutes, at times between about 2 and 8 minutes, at times between about 3 and 7 minutes or at times for about 5 minutes. According to some embodiments, the pasteurization is carried out at a temperature range of 80° C. to 90° C. for from 1 to 50 minutes. According to some embodiments, the pasteurization is followed by cooling the pasteurized composition to a temperature below 50° C., or below 40° C., or below 35° C., e.g., about 30° C. According to some embodiments, the pasteurization is followed by cooling the pasteurized composition to a temperature optimal for lactic acid bacteria growth. Thus, according to some embodiments, the pasteurization is followed by cooling the pasteurized composition to from 60 to 70° C.

According to some embodiments, the composition is homogenized at step (ii) and pasteurized at step (iii) as described in any one of the above embodiments. Any and all limitations regarding the homogenization and pasteurization provided in any one of the above embodiments are encompassed herein as well, as well as any and all of their combinations.

A person of average skill in the art would understand that step (v) provides an alternative dairy product which comprises LAB and LAB-fermentation-sufficient nutrients. A person of average skill in the art would further understand that step (vi) is optional as it can be performed independently, in separation in time and place, from step (v). For example, fermentation may be performed after filling the alternative dairy product comprising LAB into the final container.

The terms “alternative dairy product”, “dairy substitute” and “dairy alternative” are used herein interchangeably and refer to any consumable/edible product or foodstuff, which is not made from or derived from animals' milk. Such products may replace animal-based products in one's diet by having the nutritional and/or rheologic and/or organoleptic and/or physicochemical properties of the corresponding traditional animal-milk-based products.

According to some embodiments, the alternative dairy product is a non-animal alternative dairy product. The terms “non-animal” and “animal-free” refer to a product that is entirely free of animal-derived, and specifically free of milk-derived, components, such as BLG or other milk proteins. In this context, the term “milk” refers to milk from mammal animals such as cow, goat, and sheep's milk. While all components of such products are non-animal, the present invention specifically relates to products comprising at least one recombinant component or ingredient.

The term “recombinant dairy ingredient” refers to any ingredient found in mammal dairy, which is recombinantly produced. According to one embodiment, the recombinant dairy ingredient is selected from a recombinant dairy protein, a recombinant dairy fat, and a recombinant dairy carbohydrate.

According to some embodiments, the recombinant dairy ingredient is a recombinant dairy protein. According to some embodiments, the recombinant dairy protein is a recombinant whey protein. According to some embodiments, the recombinant dairy protein is β-Lactoglobulin (BLG). According to some embodiments, the recombinant dairy protein is isoform B of the BLG. The term “beta-lactoglobulin” (BLG) refers to a beta-lactoglobulin that is typically present in cow's milk. As used in the present invention, the term BLG further refers to isoform B of the BLG, i.e., beta-Lactoglobulin B (β-LG B), which is a small protein of 162 amino acids with a molecular mass of 18.2 kDa and optimum pH of 5.2 (UniProt D6QX31). Nevertheless, in some specific embodiments, the term BLG may refer to BLG-A isoform or to a combination of BLG-A and BLG-B.

According to some embodiment, the recombinant dairy protein is produced in microorganisms such as bacteria and yeasts. According to some embodiments, the recombinant dairy protein is BLG (rBLG), produced in yeasts. According to some embodiments, the recombinant dairy protein is BLG produced in Pichia pastoris and specifically BG11. According to some embodiments, the recombinant dairy protein constitutes at least 10% of all proteins in the alternative dairy product. According to some embodiments, the recombinant dairy protein constitutes at least 15%, at least 20%, at least 25%, at least 30%, at least 35%, at least 40%, at least 45, or at least 50% of all proteins of the alternative dairy product. According to some embodiments, the recombinant dairy protein constitutes at least 55 wt %, at least 60 wt %, at least 65 wt %, at least 70 wt %, at least 75 wt %, at least 80 wt %, at least 85 wt %, at least 90 wt %, at least 95 wt %, at least 97 wt %, at least 98% or at least 99% of all proteins in the alternative dairy product. According to some embodiments, the recombinant dairy protein constitutes at least 15%, at least 20%, at least 25%, at least 30%, at least 35%, at least 40%, at least 45, at least 50%, at least 55 wt %, at least 60 wt %, at least 65 wt %, at least 70 wt %, at least 75 wt %, at least 80 wt %, at least 85 wt %, at least 90 wt %, at least 95 wt %, at least 97 wt %, at least 98% or at least 99% of the total milk-protein content of the alternative dairy product. According to some embodiment, the recombinant dairy protein is rBLG.

According to some embodiments, the rBLG constitutes from about 51 wt % to about 100 wt %, from about 55 wt % to about 99 wt %, from about 60 wt % to about 98 wt %, from about 65 wt % to about 97 wt %, from about 70 wt % to about 95 wt %, from about 75 wt % to about 93 wt %, from about 80 wt % to about 90 wt %, from about 60 wt % to about 80 wt %, or from about 70% to about 90% of the total milk-protein content or of all proteins of the alternative dairy product. According to some embodiments, the rBLG constitutes from about 55 wt % to about 95 wt %, from 60 wt % to about 90 wt %, from about 65 wt % to about 85 wt %, from about 70% to about 80 wt %, from 70% to about 99 wt %, from 75 wt % to about 95 wt %, from 85% to about 95 wt %, from 90 wt % to about 100 wt % or from 95 wt % to about 99 wt % of the total milk-protein content or of all proteins of the alternative dairy product. According to some embodiment, the recombinant dairy protein is rBLG. Each range of the listed ranges represent a separate embodiment and may be used in combination with any other embodiment of the present application.

According to some embodiments, the recombinant dairy protein constitutes at least 1 wt % of the alternative dairy product. According to some embodiments, the recombinant dairy protein constitutes at least 2 wt %, at least 3 wt %, at least 4 wt %, at least 5%, at least 6 wt %, at least 8 wt % or at least 10 wt % of the alternative dairy product. According to some embodiments, the recombinant dairy protein constitutes from 1 to 20 wt %, from 2 to 18 wt %, from 3 to 16 wt %, from 4 to 14 wt %, from 5 to 12 wt %, from 6 to 10 wt %, from 1 to 12 wt %, from 2 to 10 wt %, from 3 to 8 wt %, from 2 to 8 wt % of the alternative dairy product. According to some embodiment, the recombinant dairy protein is rBLG. According to some embodiments, the recombinant dairy protein such as rBLG constitutes from 1 to 20 wt % of the alternative dairy product and constitutes from about 51 wt % to about 100 wt % of the total milk-protein content or of all proteins of the alternative dairy product.

According to some embodiments, the BLG and/or the rBLG have the amino acid sequence SEQ ID NO: 1. The term “BLG” encompasses known BLG variants, for example, known bovine BLG variants, and also analogs and chimera of the BLG. The term “analog”, “analog” and “sequence analog” are used herein interchangeably and refer to an analog of a peptide, polypeptide or protein having at least 70% sequence identity with the original peptide, wherein the analog retains the activity of the original peptide or protein. Thus, the terms “analog” and “active analog” may be used interchangeably. In some examples, the analog has at least 99%, 98%, 97%, 96% or 95% sequence identity with the original sequence. The term “analog” refers to a peptide, polypeptide or protein which contains substitutions, rearrangements, deletions, additions and/or chemical modifications in the amino acid sequence of the parent peptide. The substitutions of the amino acids may be conservative or non-conservative substitution. The non-conservative substitution encompasses substitution of one amino acid by any other amino acid. In one particular embodiment, the amino acid is substituted by a non-natural amino acid. The term “conservative substitution” as used herein denotes the replacement of an amino acid residue by another, without altering the overall conformation and biological activity of the peptide, including, but not limited to, replacement of an amino acid with one having similar properties (such as, for example, polarity, hydrogen bonding potential, acidic, basic, shape, hydrophobic, aromatic, and the like). Amino acids with similar properties are well known in the art. For example, according to one table known in the art, the following six groups each contain amino acids that are conservative substitutions for one another: (1) Alanine (A), Serine(S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W).

According to some embodiments, the BLG protein comprises an amino acid sequence selected from SEQ ID NOs: 1-10. According to some embodiments, the term BLG also encompasses analogs thereof. According to some embodiments, the BLG analog comprises an amino acid sequence selected from SEQ ID NOs: 11-20.

According to some embodiments, the recombinant dairy protein is the sole whey protein in the alternative dairy product. According to some embodiments, the recombinant dairy protein is the sole milk protein in the alternative dairy product. According to some embodiments, the recombinant dairy protein is the whey protein in the alternative dairy product. According to some embodiments, the recombinant dairy protein is the sole protein in the alternative dairy product. According to some embodiment, the recombinant dairy protein is rBLG. The term “total milk-protein content” refers to the content of all milk proteins in the composition. In the context of the present disclosure, when referring to “milk protein” or “dairy protein” it is to be understood as meaning proteins present in milk, yet not necessarily isolated from milk. In certain embodiments, the term “milk protein” refers to BLG, ALA, αS1 casein, αS2 casein, β casein, and k casein.

According to some embodiments, the recombinant dairy ingredient is a dairy fat. In certain embodiments, the recombinant dairy fat is selected from triacylglycerol, 1,2-diacylglycerol, cholesterol, cholesteryl ester, and monoacylglycerol. In certain embodiments, the recombinant dairy fat is triacylglycerol.

According to some embodiments, the recombinant dairy ingredient is lactose.

According to any one of the above embodiments, the LAB is a non-pathogenic LAB capable of acidifying milk, generating flavor, texture, and any combination of the above. Any known LAB may be used according to the teaching of the present invention. According to any one of the above embodiments, the LAB are non-pathogenic LAB capable of acidifying alternative dairy product, generating flavor, generating texture, and any combinations of the above. The LAB can be mixed-strain or defined-strain cultures. In some examples, the bacteria culture is mesophilic. In some other examples, the bacteria culture is thermophilic. According to some embodiments, the LAB is selected from Lactobacillus Bulgaricus, Streptococcus Thermophilus, Streptococcus Group N1, Leuconostoc, Lactobacillus Acidophilus, Lactobacillus Casei, Lactobacillus Paracasei, Bifidobacterium Lactis, Lactococcus lactis subsp. Lactis, Leuconostoc Mesenteroides subsp, Lactobacillus Acidophilus, and any combinations thereof. According to some embodiments, the LAB is Lactobacillus Bulgaricus. According to some embodiments, the LAB is Streptococcus Thermophilus. According to some embodiments, the LAB is a combination of Lactobacillus Bulgaricus and Streptococcus Thermophilus.

According to some embodiments, the method comprises filling the resulting alternative product into a final container after the addition of the LAB, optionally without waiting for LAB fermentation. According to some embodiments, the filling is performed after the completion of step (v), e.g., in an alternative optional step (iv). According to some embodiments, the filling of the alternative product into a final container is performed in aseptic conditions. According to some embodiments, after pasteurization, all steps are performed in aseptic conditions.

According to any one of the above embodiments, the method comprises LAB fermentation in step (vi) thereby obtaining a LAB-fermented alternative dairy product. The term “acidification” and/or “fermentation” refers to the process of reducing the pH of the composition carried out using LAB. The term “fermenting the alternative dairy product”, “LAB fermenting”, “allowing the LAB to ferment”, “initiating LAB fermentation” and “providing LAB fermentation” may be used interchangeably and refer to providing conditions such as a temperature and time, for LAB to ferment the alternative dairy product. According to some embodiments, the alternative dairy product is allowed to ferment for at least 2, at least 3, at least 4, at least 4.5, at least 6, at least 8, at least 12 hours or at least 16 hours. According to some embodiments, the alternative dairy product is fermented for from about 4.5 to about 48 hours. According to some embodiments, the alternative dairy product is fermented for from about 4.5 to about 24 hours. According to some embodiments, the alternative dairy product is fermented for from about 6 to about 16 hours, from about 6 to about 12 hours, from about 7 to about 14 hours, or from about 8 to about 12 hours. According to some embodiments, the alternative dairy product is fermented for from about 4.5 to 8 hours. According to some embodiments, the alternative dairy product is fermented at a temperature of from 30 to 45° C., from 34 to 42° C., from 35 to 40° C. or at about 37° C. According to some embodiments, the alternative dairy product is allowed to ferment for from 4.5 to 48 hours at a temperature of from 30 to 45° C. According to some embodiments, the alternative dairy product is allowed to ferment for from 6 to 16 hours at a temperature of from 35 to 40° C. According to some embodiments, the alternative dairy product is allowed to ferment for from 6 to 12 hours at a temperature of from 35 to 40° C. According to some embodiments, the method comprises fermentation for about 6, about 7, about 8, about 9, about 10, about 12, about 14 or about 16 hours at a temperature of from 35 to 40° C. According to some embodiments, the alternative dairy product is allowed to ferment until the pH reaches the desired pH, e.g., from 3.9 to 4.7.

According to some embodiments, the fermentation may be carried out in the final container. According to some embodiments, the filling of the alternative product into a final container is performed in aseptic conditions. According to some embodiments, the fermentation in the final container is performed in conditions allowing and/or facilitation LAB fermentation. According to some embodiments, the fermentation may be carried out in the final container at a temperature of from 30 to 45 or from 35 to 40° C. Considering that the method of the present invention provides faster acidification (i.e. reaching the desired pH), and faster development of dairy product flavors, according to some embodiments, the alternative product is filled into the final container about 2 hours, or about 3 hours or after 4 hours, or about 5 hours, or about 6 hours or about 7 hours after LAB is added. According to some embodiments, the alternative dairy product is left to ferment in the final container for an additional 2 to 16 hours, for additional 3 to 8 hours or for additional 4 to 8 hours, in conditions allowing and/or facilitating LAB fermentation.

According to some embodiments, the fermented alternative dairy product is filled into the final container after the completion of fermentation (e.g., when the desired pH is reached).

According to any one of the above embodiments, the method further comprises adjusting the pH of a composition to the range of from 6.4 to 7.2 at any step before adding the LAB. According to some embodiments, the method comprises adjusting the pH of a composition to from 6.6 to 7 or to about 6.8.

According to any one of the above embodiments, the method further comprises adding a coagulation mineral salt before adding the LAB. According to some embodiments, the coagulation salt is added together with the yeast composition. The terms “coagulation salt”, “coagulation mineral”, “calcium source” and “coagulation mineral salt” may be used herein interchangeably and refer to a mineral, e.g., in the form of soluble salt, or ions thereof that initiate protein coagulation, as known in the art. It is known that soluble salt upon dissolution disintegrates into ions forming it. Thus, according to some embodiments, the term salt refers also to a dissolved coagulation salt. Upon dissolution, coagulation mineral salt provides cations that initiate coagulation. According to some embodiments, the coagulation mineral comprises one or more salts of a mineral selected from calcium, magnesium, phosphorus, potassium, selenium, and zinc. In some examples, the coagulation mineral is calcium or magnesium. In some examples, the coagulation mineral salt is a calcium salt or a magnesium salt. In some examples, the coagulation mineral salt is selected from calcium chloride, magnesium chloride, and calcium lactate. According to some embodiments, the coagulation mineral salt is calcium chloride. According to some embodiments, the method comprises adding from about 0.0015 to about 0.35 wt % of the coagulation mineral salt. According to some embodiments, the method comprises adding from about 0.005 to about 0.25 wt %, from about 0.01 to about 0.20 wt % or from about 0.05 to about 0.2 wt % of the coagulation mineral salt, such as calcium chloride.

According to some embodiments, the method further comprises adding a non-animal lipid in step (i), prior to the pasteurization step. The term “non-animal lipid” refers to any lipid, fat and oil that does not originate from an animal and/or milk. According to some embodiment, the lipid is plant-derived lipid. According to some embodiments, the lipids comprise an oil. According to some embodiment, the oil is selected from shea oil, sunflower oil, coconut oil, rapeseed oil, nut oil, palm oil, kernel oil, olive oil, soya oil, cotton oil, and cocoa butter (Theobroma oil). According to some embodiments, the method comprises adding from 1 to 40 wt % of the non-animal fat. According to some embodiments, the method comprises adding from 1 to 10 wt % of the non-animal fat. According to some embodiments, the method comprises adding from 10 to 40 wt %, from 15 to 35 wt % or from 20 to 30 wt % of the non-animal fat such as plant oil. Each one of the embodiments may be combined with any embodiment of the present application.

According to some embodiments, the method further comprises adding sugar. According to some embodiments, sugar may be added in one step or in a plurality of steps. According to some embodiments, sugar is added in step (i). According to some embodiments, sugar is added after completion of the fermentation. The term “sugar” refers to any edible sugar, carbohydrate, or sugar substitute. According to some embodiments, the sugar is a non-animal sugar. According to some embodiment, the sugar is plant-derived sugar. According to some embodiments, the sugar is selected from a monosaccharide, disaccharide, and polysaccharide. According to some embodiments, the sugar is selected from glucose, fructose, mannose, xylose, arabinose, sucrose, dextrose, maltose, and galactose. According to some embodiments, the method comprises adding from 1 to 20 wt % of sugar. According to some embodiments, the method comprises adding from 1 to 5 wt % of sugar. According to some embodiments, the method comprises adding from 1 to 15 wt % of sugar. According to some embodiments, the method comprises adding from 1 to 15 wt %, from 1 to 14%, from 1 to 13%, from 1 to 12%, from 1 to 11%, from 1 to 10%, from 1 to 9%, from 1 to 8%, from 1 to 7% or from 1 to 6% of sugar. Each one of the embodiments may be combined with any embodiment of the present application.

According to some embodiments, the method further comprises adding a stabilizer. According to some embodiments, the stabilizer is added in step (i). The term “stabilizer” as used herein refers to an additive to food which helps to preserve its structure. Non-limiting examples of stabilizers are functional enzymatically treated potato starch such as Etenia 457, starch, Locust bean gum, pectin, Carrageenan, and any combination thereof. According to some embodiments, the method further comprises adding from about 0.01 to about 1 wt % of the stabilizer. Each one of the embodiments may be combined with any embodiment of the present application.

According to any one of the above embodiments, the method further comprises adding a chelating agent at step (i). The terms “chelating agent”, “chelating salt”, “chelator”, “coagulation mineral chelator” and “chelating mineral” are used herein interchangeably and refer to agents capable of chelating cations, such as divalent ions. In some embodiments, the chelating agent chelates divalent ions. In some embodiments, the chelating agent is added to chelate divalent cation(s) to prevent early or spontaneous coagulation. In some examples, the chelating agent is a sodium salt. In some examples, the chelating agent is selected from sodium citrate, trisodium citrate, sodium phosphate, and sodium orthophosphate. In some examples, the salt is in its soluble form. Each one of the embodiments may be combined with any embodiment of the present application.

In some other examples, the salt is in dry form. According to some embodiments, the method further comprises adding from about 0.01 to about 1 wt % of the chelating agent. According to some embodiments, the method further comprises adding from about 0.1 to about 0.2 wt % of the chelating agent.

It would be further understood to any person of average skill in the art that all components added at steps (i) and (iv) may be added in a single step. According to some embodiments, all components added at steps (i), (iv) and (v) are added in a single step. According to any one of the above embodiments, the composition is further homogenized and/or pasteurized.

According to any one of the above embodiments, the resulting alternative dairy product is selected from yogurt, cheese, cream cheese, buttermilk, sour cream, kefir, soured milk, Crème fraiche, and ryazhenka. According to some embodiments, the alternative dairy product is a fermented alternative dairy product. According to some embodiments, the resulting alternative dairy product is an alternative yogurt or an alternative cream cheese. According to some embodiments, the resulting alternative dairy product is an alternative yogurt. According to some embodiments, the resulting alternative dairy product is an alternative cream cheese.

According to any one of the above embodiments, the method provides an alternative dairy product or a fermented dairy product having a flavor characteristic of the corresponding traditional animal-milk-based dairy product. According to any one of the above embodiments, the method provides an alternative dairy product or a fermented dairy product with a decreased off flavor compared to a corresponding composition without the use of the yeast composition. According to any one of the above embodiments, the method provides an alternative dairy product or a fermented dairy product with pH values that are typical to the corresponding traditional animal-milk-based dairy products. According to any one of the above embodiments, the method provides an alternative dairy product or a fermented dairy product having the same rheological and/or organoleptic properties as the corresponding traditional animal-milk-based dairy products. Non-limiting examples of such properties are pH, appearance, glossiness, consistency, structure, thickness, flavor, taste, aroma, penetration strength, and overrun. The properties as described herein, and the respective similarity to corresponding traditional animal-milk-based dairy products, are achieved due to improved fermentation of LAB conferred by addition of yeast composition as provided in the present invention.

According to some embodiments, the method provides a faster decrease in pH of the fermented alternative dairy product e.g., alternative yogurt or cream cheese. According to some embodiments, the method provides reaching pH of below 4.8 after about 4 hours. According to some embodiments, the method provides reaching pH of below 4.5 after 8 hours.

According to some embodiments, the method provides a faster development of fermented alternative dairy product flavor e.g., flavor of alternative yogurt or cream cheese dairy product. According to some embodiment, the method provides reaching the yogurt sourness after about 1.5 hours. According to some embodiment, the method provides reaching the yogurt sourness after about 2 hours. According to some embodiment, the method provides reaching the yogurt sourness after about 2 hour or more. According to some embodiment, the method provides reaching the yogurt sourness after 2 hours and in less than 6 hours. According to some embodiment, the method provides reaching the yogurt flavor after about 2.5 hours. According to some embodiment, the method provides reaching the yogurt flavor after about 3 hours. According to some embodiment, the method provides reaching the yogurt flavor after about 4 hours. According to some embodiment, the method provides reaching the yogurt flavor after 3 hours and in less than 7 hours.

According to some embodiments, the method of the present invention comprises: (I) pasteurizing a composition comprising a recombinant BLG (rBLG); (II) adding from about 0.02 to about 0.08 wt % of a yeast composition to the composition of the previous step; and (III) adding the LAB, thereby producing the alternative dairy product, wherein the rBLG constitutes at least 10 wt % of all proteins of the alternative dairy product.

According to some embodiments, the method further comprises adding a non-animal lipid prior to pasteurizing in step (I). According to some embodiments, the rBLG constitutes from about 1 to about 20 wt %, from about 2 to about 18 wt %, from about 3 to about 16 wt %, from about 4 to about 14 wt %, from about 5 to about 12 wt %, from about 6 to about 10 wt %, from about 1 to about 12 wt %, from about 2 to about 10 wt %, from about 3 to about 8 wt %, from about 2 to about 8 wt % of the alternative dairy product. According to some embodiments, the method comprises addition of from about 0.02 to about 0.24 wt % of L-threonine in step (II) or step (III).

According to some embodiment, the lipid is a plant-derived lipid. According to some embodiments, the lipids comprise an oil. According to some embodiment, the oil is selected from shea oil, sunflower oil, coconut oil, rapeseed oil, nut oil, palm oil, kernel oil, olive oil, soya oil, cotton oil, and cocoa butter (Theobroma oil).

According to some embodiments, the method further comprises adding a sugar. According to some embodiments, the sugar is selected from a monosaccharide, disaccharide, and polysaccharide. According to some embodiments, the sugar is selected from glucose, fructose, mannose, xylose, arabinose, sucrose, dextrose, maltose, and galactose. According to some embodiments, the method further comprises adding a stabilizer. According to some embodiments, the stabilizer is selected from functional enzymatically treated potato starch such as Etenia 457, starch, Locust bean gum, pectin, carrageenan, and any combination thereof. According to some embodiments, the method further comprises adding a chelating agent at step (i). According to some embodiments, the chelating agent is selected from sodium citrate, trisodium citrate, sodium phosphate, and sodium orthophosphate. In some examples, the salt is in its soluble form. In some other examples, the salt is in dry form.

According to some embodiments, the method of the present invention comprises: (i) mixing a recombinant BLG (rBLG), sugar, water and optionally a stabilizer; a. adjusting the pH to the range of from 6.4 to 7; b. adding a non-animal fat; (ii) homogenizing the composition obtained in step (i); (iii) pasteurizing the composition obtained in step (ii); (iv) adding from about 0.02 to about 0.08 wt % of a yeast extract and calcium chloride; (v) adding LAB, thereby producing the alternative dairy product, wherein the rBLG constitutes at least about 10 wt % of all proteins or of all milk proteins of the alternative dairy product.

According to some embodiments, the method comprises adding from about 0.01 to about 0.24 wt % of L-threonine in step (i) or step (iv). According to any one of the above embodiments, the rBLG constitutes at least 15%, at least 20%, at least 25%, at least 30%, at least 35%, at least 40%, at least 45, or at least 50% of all proteins in the alternative dairy product. According to some embodiments, the rBLG constitutes at least 55 wt %, at least 60 wt %, at least 65 wt %, at least 70 wt %, at least 75 wt %, at least 80 wt %, at least 85 wt %, at least 90 wt %, at least 95 wt %, at least 97 wt %, at least 98% or at least 99% of all proteins in the alternative dairy product. According to some embodiments, the rBLG constitutes at least 15%, at least 20%, at least 25%, at least 30%, at least 35%, at least 40%, at least 45, at least 50%, at least 55 wt %, at least 60 wt %, at least 65 wt %, at least 70 wt %, at least 75 wt %, at least 80 wt %, at least 85 wt %, at least 90 wt %, at least 95 wt %, at least 97 wt %, at least 98 wt % or at least 99 wt % of the total milk-protein content of the alternative dairy product. According to some embodiments, the rBLG constitutes from 55 wt % to 100 wt % of the total milk-protein content of the alternative dairy product. According to some embodiments, the rBLG constitutes at least 1 wt % of the alternative dairy product. According to some embodiments, the recombinant dairy protein constitutes at least 2 wt %, at least 3 wt %, at least 4 wt %, at least 5%, at least 6 wt %, at least 8 wt % or at least 10 wt % of the alternative dairy product. According to some embodiments, the rBLG constitutes from 1 to 20 wt %, from 2 to 18 wt %, from 3 to 16 wt %, from 4 to 14 wt %, from 5 to 12 wt %, from 6 to 10 wt %, from 1 to 12 wt %, from 2 to 10 wt %, from 3 to 8 wt %, from 2 to 8 wt % of the alternative dairy product. According to some embodiments, the alternative dairy product is allowed to ferment for at least 4.5, at least 6, at least 8 or at least 12 hours at a temperature of from 30 to 45° C. According to some embodiments, the alternative dairy product is allowed to ferment for from 6 to 48 hours at a temperature of from 30 to 45° C. According to some embodiments, the alternative dairy product is allowed to ferment for from 6 to 16 hours at a temperature of from 35 to 42° C. According to some embodiments, the resulting alternative dairy product is further smoothed.

According to some embodiments, the method further comprises filling the fermented alternative dairy product into a container.

According to one embodiment, the resulting alternative dairy product is an alternative cream cheese. According to some embodiments, the method of preparing the alternative cream cheese comprises adding from about 3 to about 12 wt % of the recombinant BLG. According to some embodiments, the method of preparing the alternative cream cheese comprises adding (a) from about 15 to about 35 wt % of the non-animal lipid; and/or (b) from about 1 to about 3 wt % of the sugar; and/or (c) from about 0.1 to about 4 wt % of the stabilizer. According to some embodiments, the method of preparing the alternative cream cheese comprises adding from about 15 to about 35 wt % of the non-animal lipid, from about 1 to about 3 wt % of the sugar; and from about 0.1 to about 4 wt % of the stabilizer.

According to some embodiments, the method of preparing the alternative cream cheese comprises LAB fermenting the alternative cream cheese (a) until the pH reaches pH 4.1-4.9, or (b) for from 6 to 48 hours at a temperature of from 35 to 42° C. According to some embodiments, the method comprises fermenting the alternative cream cheese until the pH reaches from 4.2 to about 4.7. According to some embodiments, the method comprises fermenting the alternative cream cheese until the pH reaches from 4.4 to about 4.6. According to some embodiments, the alternative cream cheese is allowed to ferment for at least 4.5, at least 6, at least 8 or at least 12 hours at a temperature of from 35 to 42° C. According to some embodiments, the alternative cream cheese is allowed to ferment for from 6 to 16 hours at a temperature of from 35 to 42° C.

According to any one of the above examples, the method comprising mixing components to obtain (i) from 1% to 10% of a dairy protein; (ii) from 1% to 15% of a sugar; (iii) from 0% to 30% of a non-animal fat such as an oil; (iv) from 0.1% to 3% of a flavoring salt; (v) from 0.01% to 0.5% of a calcium source; (vi) from 0.001% to 1% of LAB; and (vii) from 0.001% to 1% of a yeast composition. According to some examples, the method comprises adding from 0.01% to 1% L-threonine. According to some examples, the dairy protein is a recombinant dairy protein. According to some examples, the dairy protein is BLG, e.g., rBLG. According to some examples, the method comprises adding from 1 to 10% BLG. According to some examples, the BLG is a sole dairy protein or a sole whey protein or a sole protein added. According to some embodiments, the method further comprises LAB fermenting the alternative dairy product obtained in step (a) using LAB, thereby obtaining a LAB-fermented alternative dairy product. According to some embodiments, the method further comprises homogenization, pasteurization or both. According to some examples, the method comprises adding (i) from 2% to 8% of a dairy protein; (ii) from 1% to 5% of a sugar; (iii) from 2% to 25% of an oil; (iv) from 0.5% to 1.5% of a flavoring salt; (v) from 0.05% to 0.25% of a calcium source; (vi) from 0.01% to 0.1% of LAB; and (vii) from 0.01% to 0.1% of a yeast composition. According to some examples, the method comprises adding from 0.02% to 0.25% L-threonine. According to some embodiments, the method comprises adding from 0.01 to 1% of trisodium citrate. According to some embodiments, the method comprises adding from 0.05 to 0.25% of trisodium citrate.

According to some embodiments, the final alternative dairy product comprises (i) from 1% to 10% of a dairy protein; (ii) from 1% to 15% of a sugar; (iii) from 0% to 30% of a non-animal fat such as an oil; (iv) from 0.1% to 3% of a flavoring salt; (v) from 0.01% to 0.5% of a calcium source; (vi) from 0.001% to 1% of LAB; and (vii) from 0.001% to 1% of a yeast composition. According to some embodiments, the final alternative dairy product comprises (i) from 2% to 8% of a dairy protein; (ii) from 1% to 5% of a sugar; (iii) from 2% to 25% of an oil; (iv) from 0.5% to 1.5% of a flavoring salt; (v) from 0.05% to 0.25% of a calcium source; (vi) from 0.01% to 0.1% of LAB; and (vii) from 0.01% to 0.1% of a yeast composition. According to some embodiments, the alternative dairy product comprises from 0.01 to 1% trisodium citrate. According to some embodiments, the alternative dairy product comprises from 0.05 to 0.25% trisodium citrate.

According to some embodiments, the present invention provides a method of preparing an alternative cream cheese, the method comprises: (i) mixing a recombinant BLG (rBLG), sugar, water and optionally a stabilizer; a. adjusting the pH to the range of from 6.4 to 7; b. adding a non-animal fat; (ii) homogenizing the composition obtained in step (i); (iii) pasteurizing the composition obtained in step (ii); (iv) adding from about 0.02 to about 0.08 wt % of a yeast extract and calcium chloride; (v) adding the LAB, thereby producing the alternative dairy product, wherein the rBLG constitutes at least about 10 wt % of all proteins or of all milk-proteins of the alternative dairy product.

According to some embodiments, the method comprises adding from about 0.01 to about 0.24 wt % of L-threonine in step (i) or step (iv). According to some embodiments, the method comprises adding from 1 to 12 wt %, from 2 to 10 wt %, from 3 to 8 wt %, from 2 to 8 wt % of rBLG in step (i). According to some embodiments, the method of preparing the alternative cream cheese comprises adding from about 15 to about 35 wt % of the non-animal lipid, from about 1 to about 3 wt % of the sugar; and from about 0.1 to about 4 wt % of the stabilizer, According to some embodiments, the method of preparing the alternative cream cheese comprises LAB fermenting the alternative cream cheese (a) until the pH reaches pH 4.1-4.9, or (b) for from 6 to 48 hours at a temperature of from 35 to 42° C. According to some embodiments, the method further comprises fermenting the alternative cream cheese until the pH reaches from 4.2 to about 4.7. According to some embodiments, the method comprises fermenting the alternative cream cheese until the pH reaches from 4.4 to about 4.6. According to some embodiments, the alternative cream cheese is allowed to ferment for at least 4.5, at least 6, at least 8 or at least 12 hours at a temperature of from 35 to 42° C. According to some embodiments, the alternative cream cheese is allowed to ferment for from 6 to 16 hours at a temperature of from 35 to 42° C. In some embodiments, yeast cells are added instead of yeast extract.

According to other embodiments, the alternative dairy product is an alternative yogurt. According to some embodiments, the method of preparing the alternative yogurt comprises adding from about 2 to about 8 wt % rBLG at step (i). According to some embodiments, the method of preparing the alternative yogurt comprises adding (a) from about 1 to about 6 wt % of the non-animal lipid; and/or (b) from about 2 to about 15 wt % of the sugar; and/or (c) from about 0.05 to about 0.3 wt % of the stabilizer. According to some embodiments, the method of preparing the alternative yogurt comprises adding from about 1 to about 6 wt % of the non-animal lipid; from about 2 to about 15 wt % of the sugar; and from about 0.05 to about 0.3 wt % of the stabilizer. According to some embodiments, the method further comprises LAB fermenting the alternative yogurt (a) until the pH reaches from 3.5 to about 4.8, or (b) for from 6 to 48 hours a temperature of from 35 to 42° C. According to some embodiments, the method comprises fermenting the alternative yogurt until the pH reaches from 3.8 to about 4.5. According to some embodiments, the method comprises fermenting the alternative yogurt until the pH reaches the value from 3.9 to about 4.4. According to some embodiments, the alternative yogurt is allowed to ferment for at least 4.5, at least 6, at least 8 or at least 12 hours at a temperature of from 35 to 42° C. According to some embodiments, the alternative yogurt is allowed to ferment for from 6 to 16 hours at a temperature of from 35 to 42° C.

According to some embodiments, the present invention provides a method of preparing an alternative yogurt, the method comprises: (i) mixing a recombinant BLG (rBLG), sugar, water and optionally a stabilizer; (a) adjusting the pH to the range of from 6.4 to 7; (b) adding a non-animal fat; (ii) homogenizing the composition obtained in step (i); (iii) pasteurizing the composition obtained in step (ii); (iv) adding from about 0.02 to about 0.08 wt % of a yeast extract and calcium chloride; (v) adding the LAB, thereby producing the alternative dairy product, wherein the rBLG constitutes at least about 10 wt % of all proteins or of all milk-proteins of the alternative dairy product.

According to some embodiments, the method comprises adding from about 0.01 to about 0.24 wt % of L-threonine in step (i) or step (iv). According to some embodiments, the method comprises adding from 1 to 12 wt %, from 2 to 10 wt %, from 3 to 8 wt %, from 2 to 8 wt % of rBLG in step (i). According to some embodiments, the method of preparing the alternative yogurt comprises adding from about 1 to about 6 wt % of the non-animal lipid; from about 2 to about 15 wt % of the sugar; and from about 0.05 to about 0.3 wt % of the stabilizer. According to some embodiments, the method comprises LAB fermenting the alternative yogurt (a) until the pH reaches from 3.5 to about 4.8, or (b) for from 6 to 48 hours a temperature of from 35 to 42° C. According to some embodiments, the method comprises fermenting the alternative yogurt until the pH reaches from 3.8 to about 4.5. According to some embodiments, the method comprises fermenting the alternative yogurt until the pH reaches the value from 3.9 to about 4.4. According to some embodiments, the alternative yogurt is allowed to ferment for at least 4.5, at least 6, at least 8 or at least 12 hours at a temperature of from 35 to 42° C. According to some embodiments, the alternative yogurt is allowed to ferment for from 6 to 16 hours at a temperature of from 35 to 42° C. In some embodiments, yeast cells are added instead of yeast extract.

According to some embodiments, the method further comprises adding yeasts at step (v). According to some embodiments, the method comprises yeast fermentation.

According to some embodiments, the present invention provides an alternative dairy product, obtainable or obtained by the method of any one of the above embodiments and aspects. According to some embodiments, the alternative dairy product comprises: (i) a recombinant dairy ingredient; (ii) a yeast-derived ingredient; and (iii) a lactic-acid-bacteria (LAB)-derived ingredient, and optionally (iv) acetaldehyde. According to some embodiments, the alternative dairy product further comprises a LAB-fermentation-related ingredient. All terms and definitions as used in any one of the above and below aspects and embodiments, apply herein as well.

According to any one of the above embodiments, the alternative dairy product is a non-animal alternative dairy product. According to any one of the above embodiments, the fermented alternative dairy product is a fermented non-animal alternative dairy product.

According to another aspect, the present invention provides an alternative dairy product, comprising (i) a recombinant dairy ingredient; (ii) a yeast-derived ingredient; and (iii) a lactic-acid-bacteria (LAB)-derived ingredient. According to some embodiments, the alternative dairy product further comprises a LAB-fermentation-related ingredient. According to some embodiments, the alternative dairy product further comprises L-threonine. According to some embodiments, the alternative dairy product further comprises acetaldehyde. All terms and definitions as used in any one of the above aspects and embodiments apply herein as well. According to some embodiments, the present invention provides an alternative dairy product comprising (i) a recombinant dairy ingredient; (ii) a yeast-derived ingredient; and (iii) a lactic-acid-bacteria (LAB)-derived ingredient and (iv) a LAB-fermentation-related ingredient. According to some embodiments, the present invention provides an alternative dairy product comprising (i) a recombinant dairy ingredient; (ii) a yeast-derived ingredient; and (iii) a lactic-acid-bacteria (LAB)-derived ingredient, (iv) a LAB-fermentation-related ingredient, and (v) acetaldehyde.

According to some embodiments, the alternative dairy product comprises a yeast-derived ingredient. According to some embodiments, the yeast-derived ingredient is selected from a yeast cell(s) such as viable cell(s), yeast DNA, yeast RNA, a yeast-derived peptide, a yeast-derived protein, a yeast-derived lipid, a yeast-derived membrane or part of it, a yeast-derived carbohydrate, a yeast metabolite, and any combination of the above. According to some embodiments, the RNA is a yeast ribosomal RNA (rRNA), such as 18S, 25S and 5.8S rRNA. According to some embodiments, the yeast-derived protein comprises yeast-related (yeast-associated) post-translational modifications. According to some embodiments, the yeast-derived protein is a yeast glycoprotein. According to some embodiments, the yeast metabolite is as defined in Yeast Metabolome Databases and described, e.g., in Ramirez-Guana M et al., (YMDB 2.0: A Significantly Expanded Version of the Yeast Metabolome Database. Nucleic Acids Res. 2017 Jan. 4; 45(D1):D440-D445. PubMed: 27899612), incorporated herein by reference in its entirety. The content of the database is encompassed herein.

According to some embodiments, the alternative dairy product comprises from about 0.001 to about 0.7 wt % of the yeast-derived ingredient. According to some embodiments, the alternative dairy product comprises from about 0.005 to about 0.5 wt %, from about 0.01 to about 0.45 wt %, from about 0.05 to about 0.4 wt %, from about 0.075 to about 0.35 wt %, from about 0.1 to about 0.3 wt % of the yeast-derived ingredient. According to some embodiments, the alternative dairy product comprises from about 0.001 to about 0.7 wt % of the yeast-derived ingredient. According to some embodiments, the alternative dairy product comprises from about 0.01 to about 0.12 wt % of the yeast-derived ingredient. According to some embodiments, the alternative dairy product comprises from about 0.012 to about 0.12 wt % of the yeast-derived ingredient. According to some embodiments, the alternative dairy product comprises from about 0.01 to about 0.1 wt %, from about 0.01 to about 0.09 wt %, or from about 0.02 to about 0.08 wt % of the yeast-derived ingredient. According to some embodiments, the alternative dairy product comprises about 0.03 wt %, about 0.04 wt %, about 0.05 wt %, about 0.06 wt %, about 0.07 wt % or about 0.08 wt % of the yeast-derived ingredient. According to some embodiments, the alternative dairy product comprises a detectable level of the yeast-derived ingredient. The amount of a yeast-derived ingredient cumulatively refers to all yeast-derived ingredients.

According to some embodiments, the alternative dairy product comprises acetaldehyde. According to some embodiments, the alternative dairy product comprises from 10 to 40 ppm of acetaldehyde. According to some embodiments, the alternative dairy product comprises from 12 to 35 ppm of acetaldehyde. According to some embodiments, the alternative dairy product comprises from 15 to 35 ppm of acetaldehyde. According to some embodiments, the alternative dairy product comprises from 15 to 30 ppm of acetaldehyde. According to some embodiments, the alternative dairy product comprises from 18 to 28 ppm of acetaldehyde. According to some embodiments, the alternative dairy product comprises from 20 to 26 ppm of acetaldehyde. According to some embodiments, the alternative dairy product comprises from 21 to 25 ppm of acetaldehyde. Each one of the embodiments may be combined with any embodiment of the present application.

According to some embodiments, the alternative dairy product comprises a yeast-derived ingredient and acetaldehyde. According to some embodiments, the alternative dairy product comprises from about 0.01 to about 0.1 wt %, from about 0.01 to about 0.09 wt %, from about 0.02 to about 0.08 wt % of the yeast-derived ingredient and from 10 to 40 ppm, from 12 to 35 ppm, from 15 to 30 ppm of from 18 to 28 ppm of acetaldehyde. According to some embodiments, the alternative dairy product comprises from about 0.02 to about 0.08 wt % of the yeast-derived ingredient and from 15 to 30 ppm of acetaldehyde. Each one of the embodiments may be combined with any embodiment of the present application.

According to some embodiments, the alternative dairy product comprises from 0.2 to 9 ppm of diacetyl. According to some embodiments, the alternative dairy product comprises from 5 to 9 ppm of diacetyl. According to some embodiments, the alternative dairy product comprises from 6 to 8 ppm of diacetyl. Each one of the embodiments may be combined with any embodiment of the present application.

According to some embodiments, the alternative dairy product comprises from about 0.01 to about 0.1 wt %, from about 0.01 to about 0.09 wt %, from about 0.02 to about 0.08 wt % of the yeast-derived ingredient; from 10 to 40 ppm, from 12 to 35 ppm, from 15 to 30 ppm of from 18 to 28 ppm of acetaldehyde; and from 0.2 to 9 ppm of diacetyl. According to some embodiments, the alternative dairy product comprises from about 0.02 to about 0.08 wt % of the yeast-derived ingredient, from 15 to 30 ppm of acetaldehyde; and from 6 to 8 ppm of diacetyl. Each one of the embodiments may be combined with any embodiment of the present application.

According to some embodiments, the alternative dairy product comprises from 1.2 to 50 ppm of acetoin. According to some embodiments, the alternative dairy product comprises from 30 to 50 ppm of acetoin. According to some embodiments, the alternative dairy product comprises from 35 to 45 ppm of acetoin. Each one of the embodiments may be combined with any embodiment of the present application.

According to some embodiments, the alternative dairy product comprises from about 0.01 to about 0.1 wt %, from about 0.01 to about 0.09 wt %, from about 0.02 to about 0.08 wt % of the yeast-derived ingredient; from 10 to 40 ppm, from 12 to 35 ppm, from 15 to 30 ppm of from 18 to 28 ppm of acetaldehyde; and from 30 to 50 ppm of acetoin. According to some embodiments, the alternative dairy product comprises from about 0.02 to about 0.08 wt % of the yeast-derived ingredient, from 15 to 30 ppm of acetaldehyde; and from 35 to 45 ppm of acetoin. Each one of the embodiments may be combined with any embodiment of the present application.

According to some embodiments, the alternative dairy product comprises from about 0.01 to about 0.1 wt %, from about 0.01 to about 0.09 wt %, from about 0.02 to about 0.08 wt % of the yeast-derived ingredient; from 10 to 40 ppm, from 12 to 35 ppm, from 15 to 30 ppm of from 18 to 28 ppm of acetaldehyde; from 0.2 to 9 ppm of diacetyl; and from 30 to 50 ppm of acetoin. According to some embodiments, the alternative dairy product comprises from about 0.02 to about 0.08 wt % of the yeast-derived ingredient, from 15 to 30 ppm of acetaldehyde; ad from 6 to 8 ppm of diacetyl; and from 35 to 45 ppm of acetoin. Each one of the embodiments may be combined with any embodiment of the present application.

According to some embodiments, the alternative dairy product comprises a LAB-derived ingredient. As used herein, the term “LAB-derived ingredient” refers to any ingredient that is derived from, originated from, extracted from or obtained from LAB and preferably may be identified as such. According to some embodiments, the LAB-derived ingredient is selected from a LAB cell, LAB DNA, LAB RNA, a LAB-derived peptide, a LAB-derived protein, a LAB-derived metabolite, LAB-derived lipid, LAB-derived membrane of part of it; and any combination thereof. According to some embodiments, the dairy product comprises live LAB cells. According to some embodiments, the RNA is rRNA, such as 16S, 23S or 5S rRNA derived from LAB. According to some embodiments, the LAB-derived protein or peptide may comprise footprints specific or unique to LAB such as modifications.

According to some embodiments, the LAB is selected from Lactobacillus Bulgaricus, Streptococcus Thermophilus, Streptococcus Group N1, Leuconostoc, Lactobacillus Acidophilus, Lactobacillus Casei, Lactobacillus Paracasei, Bifidobacterium Lactis, Lactococcus lactis subsp. Lactis, Leuconostoc Mesenteroides subsp, Lactobacillus Acidophilus, and any combinations thereof. According to some embodiments, the LAB is Lactobacillus Bulgaricus. According to some embodiments, the LAB is Streptococcus Thermophilus. According to some embodiments, the LAB is a combination of Lactobacillus Bulgaricus and Streptococcus Thermophilus. Each one of the embodiments may be combined with any embodiment of the present application.

According to some embodiments, the alternative dairy product comprises at least one LAB-fermentation-related ingredient. According to some embodiments, the LAB-fermentation-related ingredient is selected from lactic acid, a volatile compound selected from acetaldehyde, diacetyl, and acetoin; and any combination thereof.

According to some embodiments, the present invention provides an alternative dairy product, comprising comprises (i) a recombinant dairy ingredient; (ii) a yeast-derived ingredient; and (iii) a lactic-acid-bacteria (LAB)-derived ingredient, (iv) a lactic acid and (v) acetaldehyde. According to some embodiments, the present invention provides an alternative dairy product, comprising comprises (i) a recombinant dairy ingredient; (ii) a yeast-derived ingredient; and (iii) a lactic-acid-bacteria (LAB)-derived ingredient, (iv) lactic acid, (v) acetaldehyde and (vi) diacetyl, acetoin or both diacetyl and acetoin. The amounts of the components are as described in any one of the embodiments of the present application.

According to some embodiments, the recombinant dairy ingredient is a recombinant dairy protein. According to some embodiments, the recombinant dairy protein is a recombinant whey protein. According to some embodiments, the recombinant dairy protein is β-Lactoglobulin (BLG). According to some embodiments, the recombinant dairy protein is BLG B. According to some embodiments, the recombinant dairy protein constitutes at least 10% of all proteins in the alternative dairy product. According to some embodiments, the recombinant dairy protein constitutes at least 15%, at least 20%, at least 25%, at least 30%, at least 35%, at least 40%, at least 45, or at least 50% of all proteins in the alternative dairy product. According to some embodiments, the recombinant dairy protein constitutes at least 55 wt % of all proteins in the alternative dairy product. According to some embodiments, the recombinant dairy protein constitutes at least 60 wt %, at least 65 wt %, at least 70 wt %, at least 75 wt %, at least 80 wt %, at least 85 wt %, at least 90 wt %, at least 95 wt %, at least 97 wt %, at least 98 wt % or at least 99 wt % of all proteins in the alternative dairy product. According to some embodiments, the recombinant dairy protein constitutes at least 15%, at least 20%, at least 25%, at least 30%, at least 35%, at least 40%, at least 45, at least 50%, at least 55 wt %, at least 60 wt %, at least 65 wt %, at least 70 wt %, at least 75 wt %, at least 80 wt %, at least 85 wt %, at least 90 wt %, at least 95 wt %, at least 97 wt %, at least 98 wt % or at least 99 wt % of the total milk-protein content of the alternative dairy product. According to some embodiments, the recombinant dairy protein constitutes at least 1 wt % of the alternative dairy product. According to some embodiments, the recombinant dairy protein constitutes at least 2 wt %, at least 3 wt %, at least 4 wt %, at least 5%, at least 6 wt %, at least 8 wt % or at least 10 wt % of the alternative dairy product. According to some embodiments, the recombinant dairy protein constitutes from 1 to 20 wt %, from 2 to 18 wt %, from 3 to 16 wt %, from 4 to 14 wt %, from 5 to 12 wt %, from 6 to 10 wt %, from 1 to 12 wt %, from 2 to 10 wt %, from 3 to 8 wt %, from 2 to 8 wt % of the alternative dairy product. According to some embodiment, the recombinant dairy protein is rBLG. According to some embodiment, the recombinant dairy protein is rBLG. According to some embodiments, the rBLG constitutes from about 51 wt % to about 100 wt %, from about 55 wt % to about 99 wt %, from about 60 wt % to about 98 wt %, from about 65 wt % to about 97 wt %, from about 70 wt % to about 95 wt %, from about 75 wt % to about 93 wt %, from about 80 wt % to about 90 wt %, from about 60 wt % to about 80 wt %, or from about 70 wt % to about 90 wt % of the total milk-protein content or of all proteins in the alternative dairy product. According to some embodiments, the rBLG constitutes from about 55 wt % to about 95 wt %, from 60 wt % to about 90 wt %, from about 65 wt % to about 85 wt %, from about 70% to about 80 wt %, from 70% to about 99 wt %, from 75 wt % to about 95 wt %, from 85% to about 95 wt %, from 90 wt % to about 100 wt % or from 95 wt % to about 99 wt % of the total milk-protein content or of all proteins in the alternative dairy product. According to some embodiments, the alternative dairy product comprises from 1 to 20 wt %, from 2 to 18 wt %, from 3 to 16 wt %, from 4 to 14 wt %, from 5 to 12 wt %, from 6 to 10 wt %, from 1 to 12 wt %, from 2 to 10 wt %, from 3 to 8 wt %, from 2 to 8 wt % of rBLG.

According to some embodiments, the BLG comprises an amino acid sequence selected from SEQ ID NO: 1-10. According to some embodiments, the BLG comprises the amino acid sequence SEQ ID NO: 1. According to some embodiments, the term BLG also encompasses analogs thereof. According to some embodiments, the BLG analog comprises an amino acid sequence selected from SEQ ID NO: 11-20.

According to some embodiments, the recombinant dairy protein is the only protein in the alternative dairy product. According to some embodiments, the rBLG is the only dairy protein in the alternative dairy product. According to some embodiments, the rBLG is the only whey protein in the alternative dairy product. According to some embodiments, the rBLG is the only protein in the alternative dairy product.

According to some embodiments, the recombinant dairy ingredient is a recombinant dairy fat. According to some embodiments, the recombinant dairy ingredient is a recombinant dairy carbohydrate.

According to some embodiments, the alternative dairy product further comprises a non-animal lipid. According to some embodiment, the lipid is plant-derived lipid. According to some embodiments, the lipids comprise an oil. According to some embodiment, the oil is selected from shea oil, sunflower oil, coconut oil, rapeseed oil, nut oil, palm oil, kernel oil, olive oil, soya oil, cotton oil, and cocoa butter (Theobroma oil). According to some embodiments, the alternative dairy product comprises from 1 to 40 wt % of the non-animal fat. According to some embodiments, the alternative dairy product comprises from 1 to 10 wt % of the non-animal fat. According to some embodiments, the alternative dairy product comprises from 10 to 40 wt %, from 15 to 35 wt % or from 20 to 30 wt % of the non-animal fat such as plant oil. Each one of the embodiments may be combined with any embodiment of the present application.

According to some embodiments, the alternative dairy product further comprises a sugar. According to some embodiments, the sugar is selected from a monosaccharide, disaccharide, and polysaccharide. According to some embodiments, the sugar is selected from glucose, fructose, mannose, xylose, arabinose, sucrose, dextrose, maltose, and galactose. According to some embodiments, the alternative dairy product comprises from 1 to 20 wt % of sugar. According to some embodiments, the alternative dairy product comprises from 1 to 5 wt % of sugar. According to some embodiments, the alternative dairy product comprises from 1 to 15 wt % of sugar. According to some embodiments, the alternative dairy product comprises from 1 to 15 wt %, from 1 to 14%, from 1 to 13%, from 1 to 12%, from 1 to 11%, from 1 to 10%, from 1 to 9%, from 1 to 8%, from 1 to 7% or from 1 to 6% of sugar. Each one of the embodiments may be combined with any embodiment of the present application.

According to some embodiments, the alternative dairy product further comprises a stabilizer. According to some embodiments, the stabilizer is selected from functional enzymatically treated potato starch such as Etenia 457, starch, Locust bean gum, pectin, carrageenan, and any combination thereof. According to some embodiments, the alternative dairy product comprises from about 0.01 to about 1 wt % of the stabilizer. Each one of the embodiments may be combined with any embodiment of the present application.

According to some embodiments, the alternative dairy product further comprises a chelating agent. According to some embodiments, the chelating agent is selected from sodium citrate, trisodium citrate, sodium phosphate, and sodium orthophosphate. According to some embodiments, the alternative dairy product further comprises from about 0.01 to about 1 wt % of the chelating agent. According to some embodiments, the alternative dairy product further comprises from about 0.1 to about 0.2 wt % of the chelating agent. Each one of the embodiments may be combined with any embodiment of the present application.

According to any one of the above embodiments, the alternative dairy product is selected from yogurt, cheese, cream cheese, buttermilk, sour cream, kefir, soured milk, crème fraiche, and ryazhenka. According to some embodiments, the alternative dairy product is a fermented alternative dairy product. According to some embodiments, the resulting alternative dairy product is an alternative yogurt. According to some embodiments, the resulting alternative dairy product is an alternative cream cheese.

According to some embodiments, the alternative dairy product comprises (i) from 1% to 10% of a dairy protein; (ii) from 1% to 15% of a sugar; (iii) from 0% to 30% of a non-animal fat, such as an oil; (iv) from 0.1% to 3% of a flavoring salt; (v) from 0.01% to 0.5% of a calcium source; (vi) from 0.001% to 1% of LAB; and (vii) from 0.001% to 1% of a yeast composition. According to some embodiments, the alternative dairy product comprises (i) from 2% to 8% of a dairy protein; (ii) from 1% to 5% of a sugar; (iii) from 2% to 25% of an oil; (iv) from 0.5% to 1.5% of a flavoring salt; (v) from 0.05% to 0.25% of a calcium source; (vi) from 0.01% to 0.1% of LAB; and (vii) from 0.01% to 0.1% of a yeast composition. According to some embodiments, the dairy protein is a recombinant dairy protein. According to some embodiments, the dairy protein is BLG. According to some embodiments, the dairy protein is rBLG. According to some embodiments, the alternative dairy product comprises from 10 to 40 ppm acetaldehyde. According to some embodiments, the method comprises adding from 0.01 to 1% of trisodium citrate. According to some embodiments, the method comprises adding from 0.05 to 0.25% of trisodium citrate.

According to some embodiments, the alternative cream cheese comprises: (i) from about 2 to about 10 wt % rBLG; (ii) from about 1 to about 3 wt % of a sugar; (iii) from about 0.1 to about 4 wt % of a stabilizer; (iv) from about 20 to about 30 wt % of a non-animal lipid; (v) from about 0.001 to about 0.5 wt % of the yeast-derived ingredient(s); and (vi) water up to 100 wt %.

According to some embodiments, the alternative cream cheese comprises: (i) from about 2 to about 10 wt % rBLG; (ii) from about 1 to about 3 wt % of a sugar; (iii) from about 0.1 to about 4 wt % of a stabilizer; (iv) from about 20 to about 30 wt % of a non-animal lipid; (v) from about 0.001 to about 0.2 wt % of the yeast-derived ingredient(s); (vi) from 10 to 40 ppm acetaldehyde; and (vii) water up to 100 wt %.

According to some embodiments, the alternative cream cheese has pH of from about 4.2 to about 4.7, or from about 4.4 to about 4.6.

According to some embodiments, the resulting alternative dairy product is an alternative yogurt. According to some embodiments, the alternative yogurt comprises: (i) from about 2 to about 8 wt % rBLG; (ii) from about 2 to about 15 wt % of a sugar; (iii) from about 0.05 to about 0.3 wt % of a stabilizer; (iv) from about 1 to about 6 wt % of a non-animal lipid; (v) from about 0.001 to about 0.5 wt % the yeast-derived ingredient; and (vi) water up to 100 wt %.

According to some embodiments, the alternative yogurt comprises: (i) from about 2 to about 8 wt % rBLG; (ii) from about 2 to about 15 wt % of a sugar; (iii) from about 0.05 to about 0.3 wt % of a stabilizer; (iv) from about 1 to about 6 wt % of a non-animal lipid; (v) from about 0.001 to about 0.2 wt % the yeast-derived ingredient; (vi) from 10 to 30 ppm acetaldehyde; and (vii) water up to 100 wt %.

According to some embodiments, the alternative yogurt has pH of from about 3.8 to about 4.5 or from about 3.9 to about 4.4. According to some embodiments, the alternative yogurt has pH of from about 4 to about 4.3.

According to any one of the above embodiments, the alternative dairy product, such as alternative yogurt or alternative cream cheese, is a non-animal alternative dairy product.

According to some embodiments, the alternative dairy product is devoid of any animal or animal-derived ingredients or components.

According to some embodiments, the alternative dairy product comprises from 1 to 3 casein proteins. According to some embodiments, the alternative dairy product is devoid of any casein proteins.

According to some embodiments, the alternative dairy product is devoid of any whey proteins except BLG and/or rBLG. According to some embodiments, the alternative dairy product is devoid of any proteins except BLG and/or rBLG.

According to some embodiments, the alternative dairy product is devoid of a yeast-fermentation-related product such as alcohol, more specifically ethanol. The terms “substantially devoid”, “essentially devoid”, “devoid”, “does not include” and “does not comprise” may be used interchangeably and refer to a composition or product that does not include, contain, or comprise a particular component, e.g., the composition comprises less than 0.01 wt %, or less than 0.001 wt % of the component.

According to yet another aspect, the present invention provides an alternative dairy product as described in any one of the above aspects and embodiments obtainable or obtained by the method defined in any one of the above aspects and embodiments. According to some embodiments, the alternative dairy product is selected from yogurt, cheese, cream cheese, buttermilk, sour cream, kefir, soured milk, crème fraiche, and ryazhenka.

The terms “a” “an” and “the”” are used herein interchangeably and mean one or more. The term “and/or” is used to indicate one or both stated cases may occur, for example, A and/or B includes, (A and B) and (A or B). The term “or” as used herein, denotes alternatives that may, where appropriate, be combined; that is, the term “or” includes each listed alternative separately as well as their combination if the combination is not mutually exclusive.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of +/−10%, or +/−5%, +/−1%, or even +/−0.1% from the specified value.

Having now generally described the invention, the same will be more readily understood through reference to the following examples, which are provided by way of illustration and are not intended to be limiting of the present invention.

### EXAMPLES

### Materials and Methods

### Preparation of an Alternative Yogurt

An alternative yogurt was prepared as follows: 4% recombinant BLG, 0.16% trisodium citrate, 3% dextrose, and 0.15% pectin LM-106 AS-YA were added to distilled water and mixed at 400 rpm at 40° C. for 40 minutes. Then, pH was adjusted to 6.8 by titration with 1M NaOH. The solution was heated to 60° C. and 2% shea fat was added. Homogenization was performed at 250 bars using a GEA Panda Plus homogenizer, followed by pasteurization at 85° C. for 2 minutes. The test articles (specified below) and 0.07% CaCl₂) were added. Then, 0.02% of Vega Harmony (Chr. Hansen) lactic acid bacteria culture (a culture for plant-based fermentation) was added to all samples, and the samples were incubated at 37° C. for 16-18 hours. Following incubation, pH was measured, and the samples were smoothed using Turrax.

A reference sample was also prepared in the same manner and comprised 11.43% skim milk powder (corresponding to 4% of the total protein), 2% shea fat, 0.15% pectin LM-106 AS-YA).

### Evaluation of the Alternative Yogurt

To evaluate the improvement in yogurt's flavor as a result of the addition of yeast extract, taste tests were conducted (n=5-7 tasters) in which the yogurt's flavor, sourness, and off-flavor were ranked (as presented below) in comparison to the reference sample, followed by a preference question.

Scale for ranking: +2: Much stronger compared to the reference; +1: Slightly stronger compared to the reference; 0: Similar to the reference; −1: Slightly weaker compared to the reference; −2: Much weaker compared to the reference.

### Preparation of an Alternative Cream Cheese

An alternative cream cheese was prepared as follows: 6% recombinant BLG, 0.15% trisodium citrate, and 2.3% dextrose were added to distilled water and were mixed at 400 rpm at 40° C. for 40 minutes. Then, pH was adjusted to 6.8 by titration with 5M NaOH. The solution was heated to 60° C. and 22.5% shea fat was added. Homogenization was performed at 500 bars using a GEA Panda Plus homogenizer, followed by pasteurization at 85° C. for 2 minutes. The test article and 0.22% calcium lactate were added. Then, 0.05% of V-M01-N (SACCO) culture was added to all samples, and the samples were incubated at 30° C. for 16-18 hours. Following incubation, pH was measured, and the samples were smoothed using Turrax.

A reference sample was also prepared in the same manner and comprised 1.55% whey protein isolate, 5.7% micellar casein concentrate, 22.5% shea fat, and 1.9% dextrose. This composition corresponds to the content of whole milk.

### Evaluation of the Alternative Cream Cheese

To evaluate the improvement of cream cheese's flavor as a result of the addition of yeast extract, taste tests were conducted (n=5 tasters) in which the cream cheese's flavor, sourness, and off-flavor were ranked (as presented below) in comparison to the reference sample, followed by a preference question.

Scale for ranking: +2: Much stronger compared to the reference; +1: Slightly stronger compared to the reference; 0: Similar to the reference; −1: Slightly weaker compared to the reference; −2: Much weaker compared to the reference.

### Example 1. Effect of Different Supplements on Alternative Yogurt's Flavor

It was first tested whether by adding different components which may be needed for lactic acid bacteria proliferation, it will be possible to enhance LAB fermentation and subsequently improve the properties of an alternative yogurt e.g., its flavor. Thus, as a first step, the following components (defined as test articles) were added during the preparation of the alternative yogurt.

### Test Articles

1) Each of the amino acids (tested separately)—threonine (20, 120 mg/100 gr), glycine (0.08, 0.5 mg/100 gr), glutamate (0.6, 20, 150 mg/100 gr), aspartic acid (0.04, 25 mg/100 gr), arginine (50 mg/100 gr);
2) Minerals—Mg (2, 12 mg/100 gr), Mn (0.02, 1 mg/100 gr), phosphate (10, 100 mg/100 gr);
3) Vitamins—Pyridoxine (0.0042, 0.042 mg/100 gr), Thiamine (0.0045, 0.045 mg/100 gr), Niacinamid (0.008, 0.08 mg/100 gr); and/or
4) α-ketoglutarate—1.5, 50, 150, 600 mg/100 gr.

### Results

None of the tested articles 1-4, alone or in combination, enhanced the LAB fermentation, i.e., none of them improved the taste of the alternative yogurt and/or to sufficiently decreased its pH. The flavor of such supplemented yogurts did not vary from the taste of the control or of yogurt without any addition.

### Example 2. Preparing an Alternative Yogurt in the Presence of Different Yeast Extracts

Inventors prepared an alternative yogurt prepared as defined in materials and method with four different commercially available yeast extracts as test articles: NuCel® 581 PW [yeast peptone obtained by the autolysis of a selected strain of yeast, especially grown on a molasses-based media], NuCel® 545 MG, NuCel® 582 MG and NuCel® 751 MG [the latter three are primary yeast extracts obtained by the autolysis of a selected strain of yeast, especially grown on a molasses-based media. CAS number: 84604-16-0; EC number: 283-294-5]). The yeast extracts were added at different concentrations: (0=NC, 0.01%, 0.05%, 0.07%, and 0.1%).

### Results

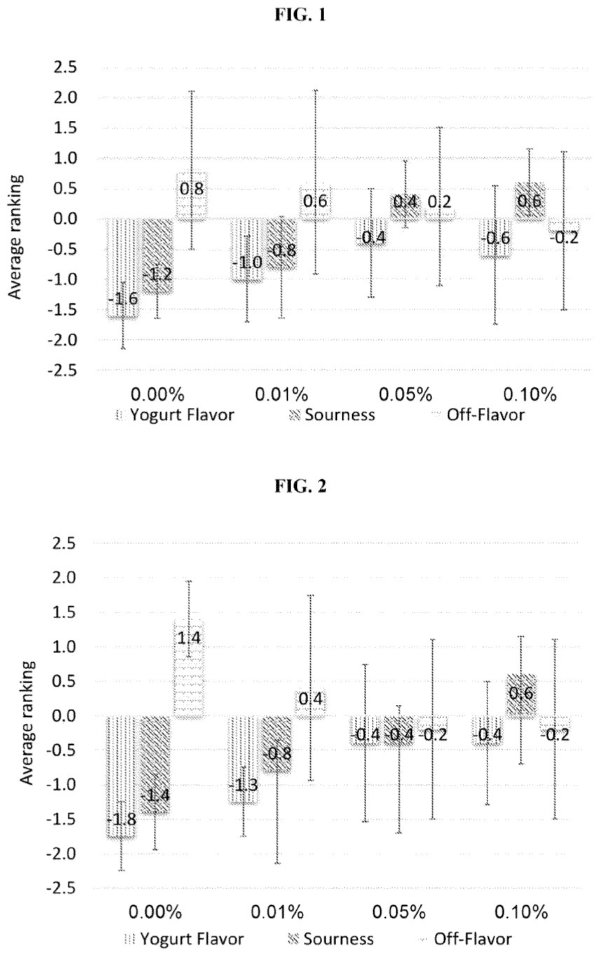

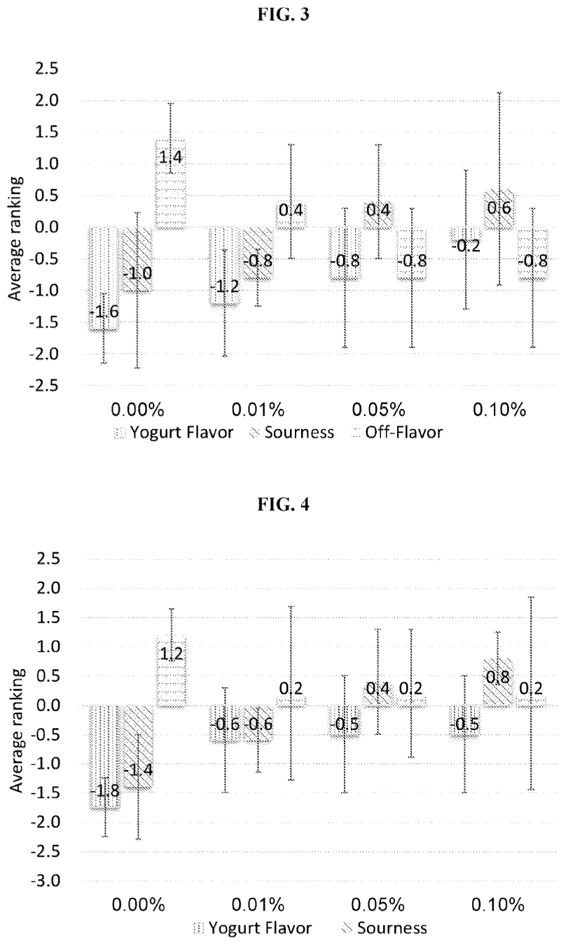

The results are presented in FIGS. 1-4. The following observations follow from these results:
Yogurt's flavor (1st indicator): According to taste test results (n=5 tasters), the addition of yeast extract led to a better yogurt flavor in all tested concentrations. The optimal concentration of each tested yeast extract in terms of preference and yogurt's parameters is between 0.01% to 0.1%.
Off-flavor (2nd indicator): According to taste test results (n=5 tasters), the addition of yeast extract led to a reduction in off-flavor.
pH after acidification (3rd indicator): After 16-18 hours of acidification at 37° C., the pH of yogurt samples supplemented with yeast extract was between 3.9 to 4.38, while the pH of the negative control sample (yogurt without yeast extract) was 4.89.

### Testing the Optimized Concentrations (0.05% and 0.07%) of Yeast Extract to Choose the Best Candidate

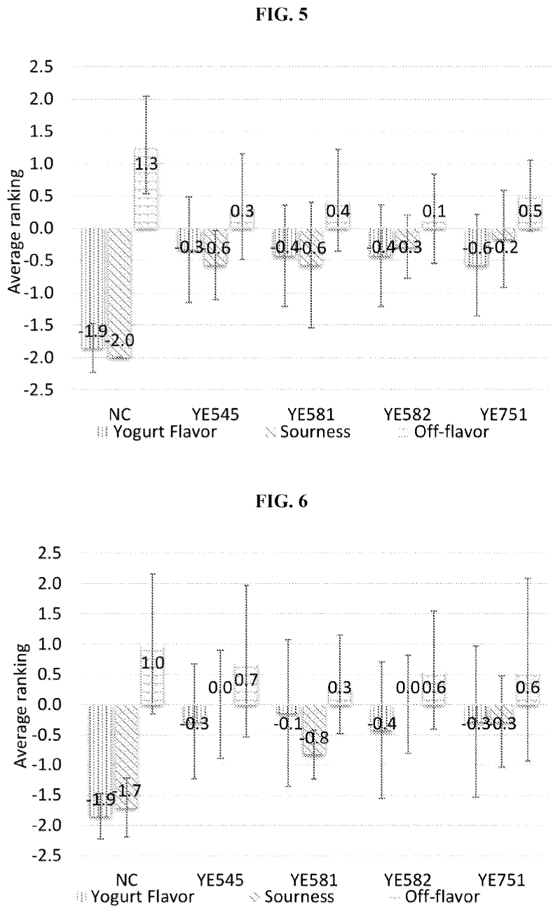

The results are presented in FIGS. 5-6. The following observations follow from these results:
Yogurt's flavor: According to taste test results (n=7 tasters), the addition of yeast extract led to a better yogurt flavor. Regarding preference, in a concentration of 0.05% yeast extract most tasters (5 out of 7) preferred the yogurt supplemented with NuCel® 582 MG, while in a concentration of 0.07% the preference was inconclusive. In 0.07% there were more off-flavors compared to 0.05%, and the tasters commented that 0.05% were better than 0.07%.
Off-flavor: According to taste test results (n=7 tasters), the addition of yeast extract led to a reduction in off-flavor. In 0.07% there were more off-flavors compared to 0.05%, and the tasters commented that 0.05% were better than 0.07%.
pH after acidification: After 16-18 hours of acidification at 37° C., the pH of yogurt samples supplemented with yeast extract was between 4.02 to 4.3, while the pH of the negative control sample (yogurt without yeast extract) was 4.78.

It can be clearly seen from these results that the addition of any yeast extract improves lactic acid bacteria fermentation which is reflected by (a) increasing characteristic yogurt's flavor, (b) decreasing off flavors, and (c) reaching the desired pH.

### Example 3. Preparing an Alternative Cream Cheese in the Presence of NuCel® 581 PW Yeast Extract

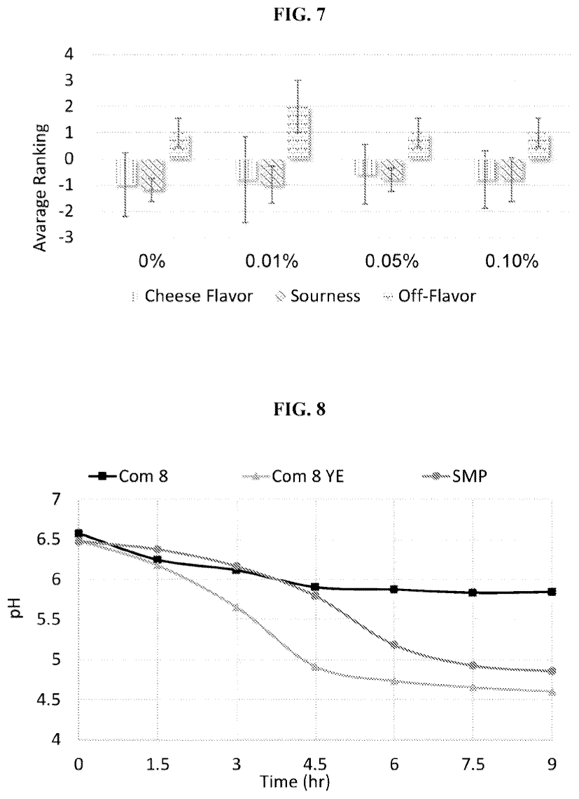

An alternative cream cheese was prepared in the presence of a commercially available yeast extract NuCel® 581 PW as defined in materials and method and compared to the control. The results of screening several concentrations of yeast extract (0, 0.01%, 0.05%, 0.1%) are presented in FIG. 7.

pH after Acidification:

After 16-18 hours of acidification at 30° C., only in cream cheese supplemented with 0.05% and 0.1% the pH reached the desired value (4.63). The pH of the negative control sample (cream cheese without yeast extract) was 5.8.

### Cream Cheese's Flavor

The results of screening several concentrations of yeast extract (0, 0.01%, 0.05%, 0.1%) are presented in FIG. 7. According to taste test results (n=5 tasters), the addition of yeast extract led to a better cream cheese flavor. The optimal concentration of yeast extract in terms of preference and cream cheese's parameters is 0.05%.

It can be clearly seen that the addition of 0.05% or more of yeast extract improves lactic acid bacteria fermentation which is reflected by the improvement in cream cheese's flavor and by reaching the desired pH.

Concluding the above, it is clear that addition of 0.01-0.1 wt % of yeast extract improved the flavor of the alternative fermented products and allowed reaching the desired pH.

### Example 4. Fermentation of an Alternative Cream Cheese Supplemented with Yeast Extract

Fermentation of an alternative cream cheese supplemented with yeast extract prepared as described in Example 3 was tested and comparted to a control. The results are presented in FIG. 8 and Table 1 below.

TABLE 1

| | pH after ON |
|---|---|
| Sample | fermentation |
| Com 8 | 5.54 |
| Com 8 + YE | 4.46 |
| SMP | 4.66 |

Com 8 represents an alternative cream cheese prepared without the addition of yeast extract (negative control), Com8 YE is the same composition as Com 8 with the addition of 0.05 wt % yeast extract, and SMP refers to skim milk powder-positive control. As can be seen from the figure, alternative cream cheese prepared in the presence of yeast extract showed the lowest pH, about 4.46 which was reached after about 7.5 hour. The other formulations reached a much higher pH. The pH reduction was not too fast what allowed development of the flavors.

### Example 5. Preparation of Alternative Step (Soft) Cream Cheese

The alternative soft cream cheese was prepared as described in Example 1 by mixing ingredients as defined in Table 2.

TABLE 2
Alternative soft cream cheese composition.

| Component | % (weight) |
|---|---|
| rBLG | 4 |
| Trisodium citrate (chelator of | 0.14 |
| calcium, mild acidic taste) | |
| Dextrose (sugar) | 2.3 |
| Starch (stabilizer) | 3 |
| Plant oil (such as shea oil) | 25 |
| NaCl | 0.9 |
| Calcium Chloride (firming agent) | 0.06 |
| Yeast extract | 0.01, 0.05 or |
| | 0.07 |
| Mesophilic lactic acid bacteria | 1 U/10 L |
| Water | Complete to |
| | 100% |

The pH of the composition was between 4.2 to 4.7. In further experiments, from about 0.02 to about 0.2 wt % (e.g., 0.02, 0.03, 0.05, 0.06, 0.1, 0.12, 0.15, 0.2 and 0.225) of L-threonine are added before lactic bacteria fermentation.

### Example 6. Preparation of an Alternative Firm Cream Cheese

The alternative firm cream cheese was prepared as described in Example 1 by mixing ingredients as defined in Table 3.

TABLE 3
Alternative firm cream cheese composition.

| Component | % (weight) |
|---|---|
| rBLG | 3.9 |
| Trisodium citrate | 0.14 |
| Dextrose (sugar) | 2.3 |
| Starch (stabilizer) | 5 |
| LBG (Locust bean gum, stabilizer) | 0.1 |
| Plant oil | 25 |
| NaCl | 0.9 |
| Calcium Chloride (firming agent) | 0.06 |
| Yeast extract | 0.01, 0.05 or 0.07 |
| Mesophilic lactic acid bacteria | 1 U/10 L |
| Water | Complete to 100% |

The pH of the composition was between 4.2 to 4.7. In further experiments, from about 0.02 to about 0.2 wt % (e.g., 0.02, 0.03, 0.05, 0.06, 0.1, 0.12, 0.15, 0.2 and 0.225) of L-threonine are added before lactic bacteria fermentation.

### Example 7. Preparation of Alternative Stirred Yogurt

The alternative stirred yogurt was prepared as described in Example 1 by mixing ingredients as defined in Table 4.

TABLE 4
Alternative stirred yogurt composition.

| Component | % (weight) |
|---|---|
| rBLG | 5.4 |
| Trisodium citrate | 0.15 |
| Dextrose (sugar) | 4 |
| Pectin (stabilizer) | 0.15 |
| Plant oil | 3.00 |
| Calcium Chloride (firming agent) | 0.07 |
| Thermophilic lactic acid bacteria | 1 U/10 L |
| Yeast extract | 0.01, 0.05 or 0.07 |
| Water | Complete to 100% |

The pH of the composition was between 3.9 to 4.4. In further experiments, from about 0.02 to about 0.2 wt % (e.g., 0.02, 0.03, 0.05, 0.06, 0.1, 0.12, 0.15, 0.2 and 0.225) of L-threonine are added before lactic bacteria fermentation.

### Example 8. Preparation of Alternative High Protein Firm Cream Cheese

The alternative high-protein firm cream cheese was prepared as described in Example 1 by mixing ingredients as defined in Table 5.

TABLE 5
Alternative high-protein firm cream cheese composition.

| Component | % (weight) |
|---|---|
| rBLG | 6.4 |
| Trisodium citrate | 0.13 |
| Dextrose (sugar) | 2.25 |
| LBG | 0.1 |
| Plant oil such as Shia oil | 22.5 |
| NaCl | 0.9 |
| Calcium Chloride (firming agent) | 0.06 |
| Mesophilic lactic acid bacteria | 1 U/10 L |
| Yeast extract | 0.01, 0.05 or 0.07 |
| Water | Complete to 100% |

The pH of the composition was between 4.2 to 4.7. In further experiments, from about 0.02 to about 0.2 wt % (e.g., 0.02, 0.03, 0.05, 0.06, 0.1, 0.12, 0.15, 0.2 and 0.225) of L-threonine are added before lactic bacteria fermentation.

### Example 9. Preparing an Alternative Yogurt in the Presence of Different Yeast Extracts and L-Threonine

### Method

Yogurt samples were prepared as follows:
1. 5% of commercial recombinant BLG, 0.1% trisodium citrate, 3.2% dextrose, and 0.02% yeast extract (NuCel® 582 MG) were added to distilled water and mixed at 400 rpm at 40° C. for 40 minutes.
2. pH was adjusted to 6.8 by titration with NaOH.
3. The solution was heated to 65° C. and 3% palm oil was added.
4. Homogenization was performed at 400 bar (2-stage homogenization) using a GEA Panda Plus homogenizer, followed by pasteurization at 85° C. for 2 minutes.
5. L-threonine was added at several concentrations (0, 0.03, 0.06, 0.12, 0.25%).
6. 0.02% of Vega Harmony (Chr. Hansen) culture was added to all samples, and the samples were incubated at 38° C. for 16-18 hours.
7. Following incubation pH was measured, and the samples were smoothed using Turrax.
8. To evaluate the improvement in yogurt's flavor following the addition of L-threonine, a taste test was conducted (n=10 tasters) in which the yogurt flavor was ranked in comparison to a reference sample (yogurt without the addition of L-threonine), followed by a preference question. The ranking scale was 0-5 with 0 representing “same as reference” and the higher the ranking means that tasters found the yogurt flavor to be stronger.
9. Finally, one concentration of L-threonine was chosen for the quantification of acetaldehyde using SPME-GC/MS method in 3 biological repeats.

### Results

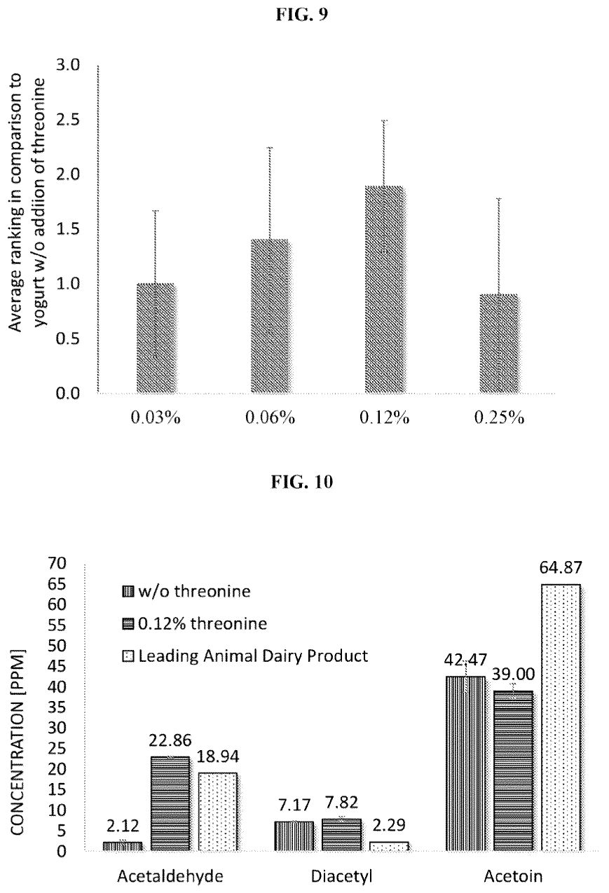

The results from the taste test are presented in FIG. 9. It can be seen, up to 0.12% of L-threonine, an increase in yogurt flavor was observed, while at 0.25% there was a decrease in the general flavor compared to the other concentrations. Moreover, several tasters commented on off-flavors and “less freshness” in the sample supplemented with 0.25% L-threonine. The chosen concentration for GC/MS analysis was 0.12%.

Acetaldehyde quantification: The addition of 0.12% L-threonine led to a 10-fold increase in acetaldehyde levels, from 2 ppm to 23 ppm. This correlates with a common convention that in a good-flavored yogurt, the levels of acetaldehyde are between 10 to 40 ppm.

Diacetyl, Acetoin and Acetic acid: Diacetyl and Acetoin are key compounds in dairy products contributing to buttery/creamy flavors. As shown in FIG. 10, addition of 0.12% L-threonine did not negatively affect the formation of diacetyl and acetoin, leaving the concentrations of these two compounds in the desired range.

A ~2.5-fold decrease in the level of acetic acid was observed in yogurt supplemented with 0.12% L-threonine (see FIG. 11). This is a positive effect since one of the undesired characteristics of alternative yogurts is their excess level of sourness.

It can be concluded that addition of L-threonine in the amount of up to 0.25% of the yogurt improves yogurt's flavor by promoting acetaldehyde formation, does not negatively affect the formation of diacetyl and acetoin, and leads to a decrease in the formation of acetic acid, which contributes to a less sour/“vinegary” taste.

### Example 10. Fermentation of an Alternative Yogurt Supplemented with Yeast Extract

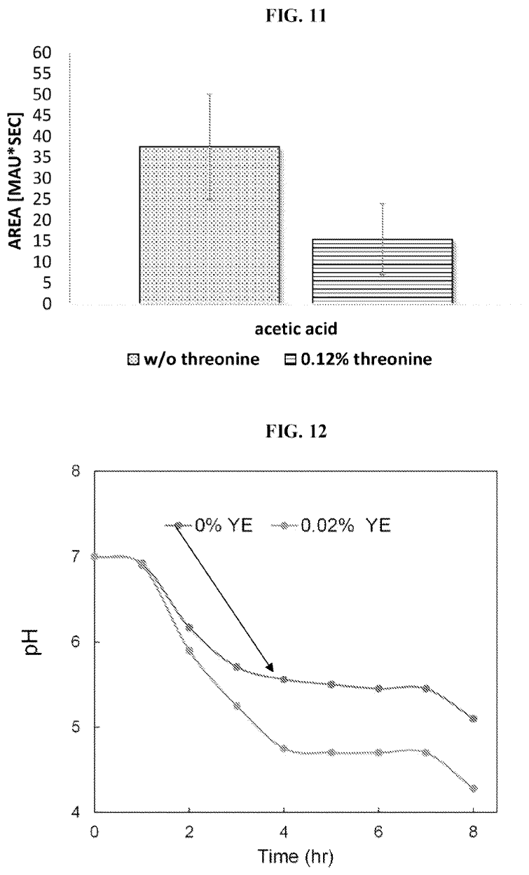

Fermentation of an alternative yogurt supplemented with 0.02% yeast extract (e.g., NuCel® 581 PW) prepared as described in Example 2 was tested and comparted to a control. [Eran, which one was used?] The results are presented in FIG. 12 and Table 6 below.

TABLE 6

| | 0% YE | | | 0.02 YE | | |
|---|---|---|---|---|---|---|
| Time | | | Yogurt | | | Yogurt |
| (Hours) | pH | Sourness | flavor | pH | Sourness | flavor |
| 0 | 7 | No | No | 7 | No | No |
| 1 | 6.92 | No | No | 6.9 | No | No |
| 2 | 6.17 | No | No | 5.9 | Yes | No |
| 3 | 5.71 | No | No | 5.25 | Yes | Yes |
| 4 | 5.56 | No | No | 4.75 | Yes | Yes |
| 5 | 5.5 | No | No | 4.7 | Yes | Yes |
| 6 | 5.45 | Yes | No | 4.7 | Yes | Yes |
| 7 | 5.45 | Yes | No | 4.7 | Yes | Yes |
| 8 | 5.1 | Yes | Yes | 4.28 | Yes | Yes |

Table 6 shows the pH reduction and taste evaluation of sourness and yogurt flavor (binary-yes or no) of yogurt without and with the addition of 0.02% yeast extract. Results show that the addition of yeast extract reduces the fermentation time to achieve sourness to about 2 hours and to achieve yogurt flavor to about 3 hours in comparison to control. It can be further seen that the pH of the supplemented yogurt was about 4.3 after 8 hours in comparison to the control in which the pH reached only 5.1.

### Example 11. Effect of the Stage of Addition of Yeast Extract

The effect of the stage of addition of yeast extracts on the properties of alternative yogurt was further explored. The alternative yogurt supplemented with 0.02% yeast extract prepared as described in Example 2 while varying the stage of adding yeast extract; yeast extract was added (1) at the hydration stage; (2) after homogenization but before pasteurization; or (3) after pasteurization.

The pH of all samples was about 4.23-4.23. A panel of five tasters were asked to taste samples 1-3 and determine if there is a difference in taste between the samples in terms of sourness and yogurt flavor. Results show that all five tasters could not distinguish between the samples. This leads to the conclusion that yeast extract may be added at any stage.

Interestingly, addition of whole yeast cells (“WYC”, Saccharomyces cerevisiae), added as dry power in the amount comparable to the amount of added yeast extract provides similar impact on pH reduction as yeast extract (“YE”) However, as follows from Table 7 below, whole yeast added before homogenization resulted in a strong umami off flavor, while addition of whole yeast cells after homogenization resulted in mild yogurt taste (if added after homogenization and pasteurization) or in a very sour taste if added after pasteurization (without homogenization). It is clear therefore that whole yeast cells may be added just as yeast extract after homogenization stage and obtain alternative dairy products such as yogurt or cream cheese with improved qualities.

TABLE 7

| | YE/ | | Final pH | | | Overall |
|---|---|---|---|---|---|---|
| | WYC | | after 16 | Sour- | Yogurt | Flavor |
| # | (0.02%) | Addition Stage | Hours | ness | Taste | Evaluation |
| 1 | WYC | Hydration | 4.1 | Yes | Yes | Umami |
| 2 | WYC | After | 4.1 | Yes | Yes | Mild yogurt |
| | | Homogenization but | | | | flavor |
| | | before pasteurization | | | | |
| 3 | WYC | After pasteurization | 4.1 | Yes | Yes | Sour |
| 4 | YE | After pasteurization | 4.3 | Yes | Yes | Good yogurt |
| | | | | | | flavor |

### Example 12. Preparation of an Alternative Cream Cheese

An alternative cream cheese was prepared as follows: 5-7% recombinant BLG, 0.15% trisodium citrate, and 2-2.5% dextrose were added to distilled water and were mixed at 400 rpm at 40° C. for 40 minutes. Then, pH was adjusted to 6.8 by titration with NaOH. The solution was heated to 60° C. and 22.5% shea fat was added. Homogenization was performed at 500 bars using a GEA Panda Plus homogenizer, followed by pasteurization at 85° C. for 2 minutes. About 0.04-0.08% of yeast extract and 0.22% calcium lactate were added. Then, 0.05% of a LAB culture (e.g., V M01 N (SACCO) culture) was added and left to incubate at 30° C. for 16-18 hours. Following incubation, pH was measured, and the product was smoothed using Turrax.

An alternative cream cheese is prepared as follows: 5-7% of recombinant BLG, 0.15% trisodium citrate, and 2-2.5% dextrose are added to distilled water and were mixed at 400 rpm at 40° C. for 40 minutes. Then, pH is adjusted to 6.8 by titration with NaOH. The solution is heated to 60° C. and 22.5% shea fat is added. Homogenization is performed at 500 bar using a GEA Panda Plus homogenizer, followed by pasteurization at 85° C. for 2 minutes. About 0.04-0.08% of whole yeast cells and 0.22% calcium lactate were added. Then, 0.05% of a LAB culture (e.g., V M01 N (SACCO) culture) is added and left to incubate at 30° C. for 16-18 hours. Following incubation, pH was measured, and the product is smoothed using Turrax.

In an additional example from about 0.02 to about 0.2 wt % (e.g., 0.02, 0.03, 0.05, 0.06, 0.1, 0.12, 0.15, 0.2 and 0.225) of L-threonine are added before lactic bacteria fermentation.

### Example 13. Preparation of an Alternative Yogurt

An alternative yogurt was prepared as follows: 4-6% of recombinant BLG, 0.13-0.17% trisodium citrate, 3-5% dextrose, and 0.15% pectin (e.g., LM-106 AS-YA) were added to distilled water and mixed at 400 rpm at 40° C. for 40 minutes. Then, pH was adjusted to 6.8 by titration with 1M NaOH. The solution was heated to 60° C. and 2-4% of plant oil such as shea fat was added. Homogenization was performed at 250 bars using a GEA Panda Plus homogenizer, followed by pasteurization at 85° C. for 2 minutes. About 0.04-0.08% of yeast extract and 0.07% CaCl₂) were added. Then, 0.02% of LAB culture (e.g., Vega Harmony (Chr. Hansen) lactic acid bacteria culture a culture for plant-based fermentation) was added and left to incubate at 37° C. for 16-18 hours. Following incubation, pH was measured, and the product was smoothed using Turrax. In an alternative example, from about 0.02 to about 0.2 wt % (e.g., 0.02, 0.03, 0.05, 0.06, 0.1, 0.12, 0.15, 0.2 and 0.225) of L-threonine were added before lactic bacteria fermentation.

An alternative yogurt is prepared as follows: 4-6% of recombinant BLG, 0.13-0.17% trisodium citrate, 3-5% dextrose, and 0.15% pectin (e.g., LM-106 AS-YA) are added to distilled water and mixed at 400 rpm at 40° C. for 40 minutes. Then, pH is adjusted to 6.8 by titration with 1M NaOH. The solution is heated to 60° C. and 2-4% of plant oil such as shea fat is added. Homogenization was performed at 250 bar e.g., using a GEA Panda Plus homogenizer, followed by pasteurization at 85° C. for 2 minutes. About 0.04-0.08% of whole yeast cells and 0.07% CaCl₂) are added. Then, 0.02% of LAB culture (e.g., Vega Harmony (Chr. Hansen) lactic acid bacteria culture a culture for plant-based fermentation) is added and left to incubate at 37° C. for 16-18 hours. Following incubation, pH is measured, and the product is smoothed using Turrax. In an alternative example, from about 0.02 to about 0.2 wt % (e.g., 0.02, 0.03, 0.05, 0.06, 0.1, 0.12, 0.15, 0.2 and 0.225) of L-threonine are added before lactic bacteria fermentation.

In yet another example, the yogurt was filled into the final container after about 2-8 hours and the fermentation was completed in the final container.

Although the present invention has been described herein above by way of preferred embodiments thereof, it can be modified, without departing from the spirit and nature of the subject invention as defined in the appended claims.

1. A method comprising the steps of:
(a) producing a composition comprising a recombinant dairy ingredient, a yeast composition and lactic-acid-bacteria (LAB), thereby obtaining an alternative dairy product; and
(b) optionally, LAB-fermenting the alternative dairy product obtained in step (a), thereby obtaining a LAB-fermented alternative dairy product.

2. The method of claim 1, wherein step (a) comprises the steps of:
(i) providing a composition comprising a recombinant dairy ingredient;
(ii) optionally, homogenizing the composition of step (i);
(iii) optionally, pasteurizing the composition of step (i) or step (ii), if present;
(iv) adding a yeast composition to the composition obtained in any one of the preceding steps; and
(v) adding lactic-acid-bacteria (LAB) to the composition obtained in step (iv), thereby obtaining an alternative dairy product.

3. The method of claim 1, wherein the yeast composition comprises at least one of:
(i) a yeast-derived amino-acid, peptide, or protein;
(ii) a yeast-derived carbohydrate;
(iii) a yeast-derived lipid;
(iv) a yeast-derived vitamin;
(v) a yeast-derived mineral; and
(vi) yeast cells.

4. (canceled)

5. The method of claim 1, wherein the yeast composition is selected from the group consisting of a yeast extract, the soluble fraction of yeast cells, yeast cells, and any combination thereof.

6. The method of claim 1, wherein the yeast composition constitutes from about 0.001 to about 1 wt % of the alternative dairy product or the LAB-fermented alternative dairy product.

7. The method of claim 1, further comprising adding L-threonine at step (a) or at any one of the steps (i)-(v).

8. The method of claim 7, comprising adding from about 0.01 to about 0.22 wt % of L-threonine.

9. The method of claim 1, wherein the recombinant dairy ingredient is selected from the group consisting of:
(i) a recombinant dairy protein;
(ii) a recombinant dairy fat; and
(iii) a recombinant dairy carbohydrate.

10. (canceled)

11. (canceled)

12. (canceled)

13. The method of claim 9, wherein the recombinant dairy protein is β-Lactoglobulin (BLG).

14. (canceled)

15. (canceled)

16. (canceled)

17. The method of claim 1, wherein the method is for at least one of:
(i) enabling or improving LAB fermentation of an alternative dairy product;
(ii) improving the flavor of a fermented alternative dairy product;
(iii) increasing the sourness of a fermented alternative dairy product;
(iv) decreasing the pH of a fermented alternative dairy product;
(v) reducing the off flavor of a fermented alternative dairy product;
(vi) shortening the acidification time of a fermented alternative dairy product;
(vii) increasing the acetaldehyde level of a fermented alternative dairy product; and/or
(viii) decreasing the acetic acid level of a fermented alternative dairy product.

18. (canceled)

19. (canceled)

20. The method of claim 1, wherein the alternative dairy product is selected from the group consisting of yogurt, cheese, cream cheese, buttermilk, sour cream, kefir, soured milk, Crème fraiche, and ryazhenka.

21. The method of claim 20, wherein the alternative dairy product is yogurt or cream cheese.

22. An alternative dairy product, obtainable or obtained by the method of claim 1.

23. (canceled)

24. An alternative dairy product, comprising:
(i) a recombinant dairy ingredient;
(ii) a yeast-derived ingredient;
(iii) a lactic-acid-bacteria (LAB)-derived ingredient; and
(iv) optionally, at least one of: a LAB-fermentation-related ingredient, L-threonine, and acetaldehyde.

25. (canceled)

26. (canceled)

27. (canceled)

28. (canceled)

29. (canceled)

30. The alternative dairy product of claim 24, comprising from 10 to 40 ppm of acetaldehyde.

31. The alternative dairy product of claim 24, wherein the recombinant dairy ingredient is selected from the group consisting of:
(i) a recombinant dairy protein;
(ii) a recombinant dairy fat; and
(iii) a recombinant dairy carbohydrate.

32. (canceled)

33. (canceled)

34. The alternative dairy product of claim 31, wherein the recombinant dairy protein is the only dairy protein in the alternative dairy product.

35. (canceled)

36. The alternative dairy product of claim 24, wherein the alternative dairy product is selected from the group consisting of yogurt, cheese, cream cheese, buttermilk, sour cream, kefir, soured milk, crème fraiche, and ryazhenka.

37. The alternative dairy product of claim 36, wherein the alternative dairy product is an alternative cream cheese or an alternative yogurt.

38. The alternative dairy product of claim 22, comprising:
(i) a recombinant dairy ingredient;
(ii) a yeast-derived ingredient;
(iii) a lactic-acid-bacteria (LAB)-derived ingredient; and
(iv) optionally, at least one of: a LAB-fermentation-related ingredient, L-threonine, and acetaldehyde.


SEQUENCE LISTING (20 sequences)

SEQ ID NO: 1 - protein, 162 aa, synthetic construct
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 2 - protein, 162 aa, synthetic construct
LIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 3 - protein, 162 aa, synthetic construct
LIVTQTMKGLDIQKVAGTWYSLAMAASDISLLDAQSAPLRVYVEELKPTPEGDLEILLHKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 4 - protein, 162 aa, synthetic construct
LIVTQTMKGLDIQKVAGTWYSLAMAASDISLLDAQSAPLRVYVEQLKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 5 - protein, 162 aa, synthetic construct
LIVTQTMKGLDIQKVAGTWYSLAMAASNISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 6 - protein, 162 aa, synthetic construct
LIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEGQCHI

SEQ ID NO: 7 - protein, 162 aa, synthetic construct
LIVTQTMKGLDIQKVAGTWYSLAMAASDISLLDAQSAPLRVYVEELKPTSEGDLEILLQKWENDECAQKKIIAEKTKIPAVFKIDALNENKVLVLDTDYKKYLLFCMENSAEPEQSLVCQCLVRTPEVYYEALEKFDKALKALPMHIRLSFNPTQLEEQCHI

SEQ ID NO: 8 - protein, 162 aa, synthetic construct
LIVTQTMKGLDIQKVAGTWYSLAMAASDISLLDAQSAPLRVYVEELKPTPEGDLEILLQKWENDECAQKKIIAEKTKMPAVFKIDALNENKVLVLDTDYKKYLLFCMENSAEPEQSLVCQCLVRTPEVDDEALEKFDKALKALPMHIRLSFNPTQLEGQCHI

SEQ ID NO: 9 - protein, 162 aa, synthetic construct
LIVTQTMKGLDIQKVAGTWYSLAMAASDISLLDAQSAPLRVYVEELKPTPEGDLELLLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 10 - protein, 162 aa, synthetic construct
LIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMGNSAEPEQSLVCQCLVRTPEVDDEALEKFDKALKALPMHIRLSFNPTQLEEQCHI

SEQ ID NO: 11 - protein, 162 aa, synthetic construct
LIVTQTMKGLDIQKVAGTWYSLAMAASDISLLDAQSAPLRVYVEELKPTPEGDLEILLHKWENGECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 12 - protein, 162 aa, synthetic construct; identical to SEQ ID NO: 3

SEQ ID NO: 13 - protein, 162 aa, synthetic construct; identical to SEQ ID NO: 8

SEQ ID NO: 14 - protein, 162 aa, synthetic construct; identical to SEQ ID NO: 9

SEQ ID NO: 15 - protein, 162 aa, synthetic construct
LIVTQTMKGLDIQKVAGTWYSLAMAASNISLLDAQSAPLRVYVEELKPTPEGDLEILLHKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 16 - protein, 162 aa, synthetic construct
LIVTQTMKGLDIQKVAGTWYSLAMAASDISLLDAQSAPLRVYVEELKPTPEGDLEILLHKWENDECAQKKIIAEKTKMPAVFKIDALNENKVLVLDTDYKKYLLFCMENSAEPEQSLVCQCLVRTPEVDDEALEKFDKALKALPMHIRLSFNPTQLEGQCHI

SEQ ID NO: 17 - protein, 162 aa, synthetic construct
LIVTQTMKGLDIQKVAGTWYSLAMAASDISLLDAQSAPLRVYVEELKPTPEGDLELLLHKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 18 - protein, 162 aa, synthetic construct
LIVTQTMKGLDIQKVAGTWYSLAMAASNISLLDAQSAPLRVYVEELKPTPEGDLEILLQKWENDECAQKKIIAEKTKMPAVFKIDALNENKVLVLDTDYKKYLLFCMENSAEPEQSLVCQCLVRTPEVDDEALEKFDKALKALPMHIRLSFNPTQLEGQCHI

SEQ ID NO: 19 - protein, 162 aa, synthetic construct
LIVTQTMKGLDIQKVAGTWYSLAMAASNISLLDAQSAPLRVYVEELKPTPEGDLELLLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 20 - protein, 162 aa, synthetic construct
LIVTQTMKGLDIQKVAGTWYSLAMAASDISLLDAQSAPLRVYVEELKPTPEGDLELLLQKWENDECAQKKIIAEKTKMPAVFKIDALNENKVLVLDTDYKKYLLFCMENSAEPEQSLVCQCLVRTPEVDDEALEKFDKALKALPMHIRLSFNPTQLEGQCHI